(19) United States
(12) Patent Application Publication
(10) Pub. No.: US 2018/0306797 A1
(43) Pub. Date: Oct. 25, 2018

(54) METHODS OF DETECTING CERAMIDE

(71) Applicants: Washington University, St. Louis, MO (US); Boston University, Boston, MA (US)

(72) Inventors: Jean E. Schaffer, St. Louis, MO (US); Daniel S. Ory, St. Louis, MO (US); Linda R. Peterson, St. Louis, MO (US); Xuntian Jiang, St. Louis, MO (US); Vasan S. Ramachandran, Boston, MA (US); Vanessa Xanthakis, Boston, MA (US); Meredith S. Duncan, Boston, MA (US)

(21) Appl. No.: 15/961,479

(22) Filed: Apr. 24, 2018

Related U.S. Application Data
(60) Provisional application No. 62/489,285, filed on Apr. 24, 2017.

(51) Int. Cl. G01N 33/574; G16H 50/30; G16H 10/60; G16H 50/20

(52) U.S. Cl. CPC G01N 33/57438; G16H 50/20; G16H 10/60; G16H 50/30

(57) ABSTRACT

Among the various aspects of the present disclosure is the provision of a method of detecting ceramides as prognostic indicators. An aspect of the present disclosure provides for a prognostic indicator for risk of developing a cardiovascular-related disease, disorder, or condition and mortality risk.

### CROSS REFERENCE TO RELATED APPLICATIONS

This application claims the benefit of U.S. Provisional Application No. 62/489,285, filed Apr. 24, 2017, the disclosure of which is hereby incorporated by reference in its entirety.

### GOVERNMENTAL RIGHTS

This invention was made with government support under grant number HL113444 awarded by National Institutes of Health. The government has certain rights in the invention.

### FIELD OF THE INVENTION

The present disclosure generally relates to methods of detecting ceramide as a prognostic indicator.

### BACKGROUND OF THE INVENTION

Cardiovascular disease (CVD) is the leading cause of death in the developed world. Clinical manifestations of CVD include myocardial infarction (MI), stroke, sudden death, angina, and heart failure (HF). Epidemiological studies have identified risk factors for CVD, including hypercholesterolemia, smoking, hypertension, diabetes and positive family history of early myocardial infarction. Nonetheless, cardiovascular events can occur in patients without these risk factors, and cardiovascular events can occur in patients who are aggressively treated for established CVD. Because CVD can be fatal upon first presentation, effective primary prevention of CVD requires identification of significant risk factors, followed by intervention to modify risk. This strategy has been very effective with respect to hypercholesterolemia, which can be detected by a blood test—a fasting lipid profile. The risk can be mitigated by employing drug therapy with a cholesterol-lowering agent, such as a statin.

in addition to cholesterol other lipids have been proposed to lead to cardiac dysfunction. One lipid class proposed to be toxic are ceramides, which have been implicated in inflammatory and apoptotic pathways. Several studies have observed increased incidence of adverse events with increasing total ceramide levels.

There are contributors to CVD independent of traditional risk factors. Cardiovascular events can occur in patients without traditional risk factors, and cardiovascular events can occur in patients who are aggressively treated for established CVD risk factors. Thus, new prognostic indicators are needed.

### SUMMARY OF THE INVENTION

Among the various aspects of the present disclosure is the provision of a method of detecting ceramides as prognostic indicators.

In one aspect, the disclosure provides a method of measuring levels of long chain ceramides and very long chain ceramides in a biological sample. In some embodiments, reduced ratios of very long chain to long chain ceramides indicate increased risk of cardiovascular related diseases, disorders, and conditions, increased risk of death from pancreatic cancer or all-cause mortality. In other embodiments, elevated levels of very long chain ceramides and lower ratios of very long chain to long chain ceramides indicate increased risk of cardiovascular mortality, or all-cause mortality.

In another aspect, the disclosure provides for a method of determining a sum of weighted expression values of a plurality of ceramides. In some embodiments, the method includes obtaining a biological sample from a subject; measuring a level of C16:0 and C22:0 or C16:0 and C24:0; or determining whether a ratio of C22:0 to C16:0 or C24:0 to C16:0 is increased or decreased compared to a reference value.

In still another aspect, the disclosure provides a method to determine if a subject is at risk of cardiovascular disease (CVD), disorders, or conditions or if a subject is at risk of death from pancreatic cancer, comprising: measuring an amount of C16:0 and C22:0 or C16:0 and C24:0 in a biological sample obtained from a subject; determining if a ratio of C22:0 to C16:0 or a ratio of C24:0 to C16:0 is increased or decreased relative to a reference value; and classifying the subject as having an increased risk of death or developing a cardiovascular disease, disorders, or conditions if the ratio of C22:0 to C16:0 or C24:0 to C16:0 is decreased relative to the reference value. In some embodiments, the subject is classified as having an increased risk of death from pancreatic cancer or all-cause mortality if the ratio of C22:0 to C16:0 or C24:0 to C16:0 is decreased relative to the reference value.

In another aspect, the disclosure provides a method to prevent cardiovascular disease (CVD), disorders, or conditions in a subject comprising: measuring an amount of C16:0 and C22:0 or C16:0 and C24:0 in a biological sample obtained from a subject; determining if a ratio of C22:0 to C16:0 or C24:0 to C16:0 is increased or decreased relative to a reference value; classifying the subject as at risk for CVD if the ratio of C22:0 to C16:0 or C24:0 to C16:0 is decreased or lower relative to the reference value; and treating the subject to prevent future CVD events when the subject is at risk for CVD.

In still yet another aspect, the disclosure provides a method for monitoring cardiovascular disease (CVD), disorders, or conditions in a subject comprising: measuring an amount of C16:0 and C22:0 or C16:0 and C24:0 in a biological sample obtained from a subject; and then at a later time, measuring an amount of C16:0 and C22:0 or C16:0 and C24:0 in a biological sample obtained from the subject, wherein a change in the ratio of C22:0 to C16:0 or C24:0 to C16:0, indicates a change in risk of the subject over time.

In some embodiments, a cardiovascular disease, disorders, or conditions comprises a cardiovascular disease (CVD), cardiac heart disease (CHD), or heart failure; or death comprises cardiovascular mortality, non-cardiovascular mortality, CVD mortality, death from pancreatic cancer or all-cause mortality.

In some embodiments, a decreased C24:0 to C16:0 ratio comprises a C24:0 to C16:0 ratio less than 13.8; or a C24:0 to C16:0 ratio less than a reference value. In some embodiments, a decreased C24:0 to C16:0 ratio indicates increased risk of cardiovascular disease, coronary heart disease, heart failure, cardiovascular mortality, non-cardiovascular mortality, increased risk of death from pancreatic cancer or all-cause mortality; or indicates increased coronary risk factors, optionally, indicates age and smoking status. In some embodiments, the reference value is a healthy human subject C24:0 to C16:0 ratio value.

In some embodiments, a decreased C22:0 to C16:0 ratio comprises a C22:0 to C16:0 ratio less than 3.7. In some embodiments, a decreased C22:0 to C16:0 ratio is compared to a reference value. In some embodiments, a decreased C22:0 to C16:0 ratio indicates the subject has an increased risk of cardiovascular disease, coronary heart disease, heart failure, cardiovascular mortality, non-cardiovascular mortality, all-cause mortality, increased systolic blood pressure, or increased total/HDL cholesterol.

In some embodiments, the C16:0 ceramide is d18:1/C16:0, the C22:0 ceramide is d18:1/C22:0, and/or the C24:0 ceramide is d18:1/C24:0.

In some embodiments, the ceramide levels are measured using tandem mass spectroscopy comprising control samples; a total number of control samples comprise at least 5% of unknown subject samples; the biological sample comprises a blood or plasma sample; the biological sample is collected from a fasting subject; the risk of cardiovascular death or non-cardiovascular death is determined; or a therapeutic intervention is performed if a ceramide ratio level indicates an increased risk of a cardiovascular disease, disorder, or condition or death.

In some embodiments, the reference value can be 13.8 or less for the C24 to C16 ratio or 3.7 or less for the C22 to C16 ratio or the reference value for C24/C16 or C22/C16 can be obtained from one or more healthy subjects.

In some embodiments, the method includes obtaining morning fasting plasma from at least one healthy control subject or at least 24 healthy control subjects; determining a range and mean values for a healthy subject at one or more time points; calculating percent change per healthy subject; or calculating Student's one sample t-test to determine if mean percent change differed from zero.

In some embodiments, method does not comprise assessing the risk of coronary heart disease (CHD) in a male subject with a decreased C24:0/C16:0 ratio.

In some embodiments, the one or more healthy control subjects comprise at least 24 healthy subjects wherein the healthy subjects are non-smoking men or women, 40 to 60 years of age, free of hypertension, free of diabetes (optionally, hemoglobin A1c<6.5% and normal glucose tolerance test), free of cardiovascular disease (optionally, normal stress echocardiogram), or free of other major systemic illness.

In some embodiments, for every standard deviation increase in plasma C24:0/C16:0 ratio, there is about a 20% lower risk of developing clinical CHD; or about a 36% lower risk of all-cause mortality.

In some embodiments, decreased levels of C22:0/C16:0 ceramide ratio indicate an increased risk for all-cause mortality; or decreased levels of C24:0/C16:0 ceramide ratio indicate and increased risk of developing CVD and prevalent CVD.

In some embodiments, decreased ratio of C24 to C16 indicates increased rate of CHD, HF, or all-cause mortality; decreased ratio of C24 to C16 or C24 to C16 indicates an increased rate of CVD-related mortality or non-CVD related mortality; elevated levels of very long chain ceramides and lower ratios of very long chain to long chain ceramides are associated with a greater incidence of cardiovascular mortality and all-cause mortality; or increased plasma C16:0 ceramide, decreased C24:0 ceramide, and decreased C24:0 to C16:0 ceramide ratios indicate increased risk of CHD and cardiovascular mortality.

In some embodiments, the biological sample is about 50 μL of plasma.

In some embodiments, the method comprises quantifying C16:0, C22:0 ceramide, or C16:0 and C24:0 ceramide with a liquid chromatography-tandem mass spectrometer (LC-MS/MS); simultaneously quantifying the most abundant circulating very long chain ceramides; or a control sample, wherein the control sample or control standard comprises at least 5% of that of subject samples.

In some embodiments, the method includes aliquoting a standard, a control, a blank, or a subject sample into a well plate; providing an internal standard/protein precipitation solution of d5-C16:0, d4-C22:0, or d4-C24:0 ceramide in isopropanol-chloroform; providing a blank comprising isopropanol-chloroform; vortexing a plate for about 3 min; centrifuging the plate for about 10 min at about 3000 g; removing a supernatant from the centrifuged plate; transferring a measured amount of the supernatant to a well plate; and providing a LC/MS spectrometer.

In some embodiments, determining a reference value comprises: quantifying C16:0, C22:0 and C24:0 ceramides with a liquid chromatography-tandem mass spectrometry (LC-MS/MS); and calculating ratios of C24:0/C16:0 and C22:0/C16:0 ceramides in the plasma of a healthy subject.

Other objects and features will be in part apparent and in part pointed out hereinafter.

### BRIEF DESCRIPTION OF THE FIGURES

The patent or application file contains at least one drawing executed in color. Copies of this patent or patent application with color drawing(s) will be provided by the Office by request and payment of the necessary fee.

FIG. 1 is a schematic representation of the 2D-LC-MS/MS system. The 2D-LC-MS/MS system includes two six-port valves (valve 1 and 2), 4 binary pumps (A, B, C, D), a HILIC column as first dimension column, a reversed phase column as second dimension column, an autosampler, and mass spectrometer (MS). Pumps A and B were used for first dimension chromatography, and pumps C and D for second dimension chromatography. When valve 1 was at position A, the sample was injected to first dimension; chromatography took place on the first dimension while the second dimension was being equilibrated for the transfer. The unwanted eluent from first dimension was diverted into waste. When valve 1 was changed to position B, the desired plug of eluent from the first dimension was transferred onto the second dimension. Valve 1 was then returned to the position A and the separation on the second dimension took place, and the first dimension was cleaned and equilibrated. Valve 2 controlled the eluent from the second dimension to waste (position A) or MS (position B).

FIG. 2 is a chart illustrating the generation of FHS samples. From 2,812 participants in the Offspring Cohort who attended their 8th examination cycle, 4 participant samples were created based on availability of plasma samples and covariate data. For Sample 1, individuals were excluded if they were missing ceramide values or covariates. For Samples 2 and 3, individuals with prevalent coronary heart disease (CHD, sample 2) and heart failure (HF, sample 3) were excluded. For Sample 4, individuals with missing follow-up time were excluded.

FIG. 3 is a chart illustrating the generation of SHIP samples. From 3,300 participants who attended SHIP-1, 4 participant samples were created based on availability of plasma samples and covariate data. For Sample A, individuals were excluded if they were missing ceramide values or covariate data. Samples B and C included those who had ceramide and covariate data and also attended SHIP-2 when CHD and HF were assessed (Sample B excluded those with CHD at SHIP-1 and Sample C excluded those with HF at SHIP-1). Sample D excluded those with missing ceramides or covariates or with unknown death date.

FIG. 4A, FIG. 4B, FIG. 4C, FIG. 4D, FIG. 4E and FIG. 4F depict the distributions of plasma ceramides in FHS and SHIP. Plots display distribution of values for C24:0 (FIG. 4A, FIG. 4D), C22:0 (FIG. 4B, FIG. 4E), and C16:0 (FIG. 4C, FIG. 4F) ceramides in FHS participants at examination 8 and in SHIP participants at SHIP-1 examination.

FIG. 5A, FIG. 5B, and FIG. 5C show cumulative incidence of Coronary Heart Disease, Heart Failure, and All-Cause Mortality in FHS by Tertiles of Plasma C24:0/C16:0 Ceramide Ratio in FHS. Cumulative incidence of coronary heart disease (CHD, FIG. 5A), heart failure (HF, FIG. 5B), and all-cause mortality (FIG. 5C) are reported for tertiles of C24:0/C16:0 ceramide ratio. Tertile 1 includes participants with ceramide levels the 33rd percentile [2.8, 12.3]; tertile 2 includes participants with ceramide levels >the 33rd and <66th percentile [12.3, 15.1]; tertile 3 includes participants with ceramide levels the 66th percentile [15.1, 29.2].

FIG. 6A, FIG. 6B and FIG. 6C show cumulative incidence of Coronary Heart Disease (CHD), Heart Failure (HF), and All-Cause Mortality in FHS by Tertiles of Plasma C22:0/C16:0 Ceramide Ratios in FHS. Cumulative incidence of coronary heart disease (CHD, FIG. 6A), heart failure (HF, FIG. 6B), and all-cause mortality (FIG. 6C) are reported for tertiles of C22:0/C16:0 ceramide ratio. Tertile 1 includes participants with ceramide levels ≤the 33rd percentile [1.0, 3.4]; tertile 2 includes participants with ceramide levels between the 33rd and 66th percentile [3.4, 4.1]; tertile 3 includes participants with ceramide levels ≥the 66th percentile [4.1, 10.5].

FIG. 7 shows risk of Coronary Heart Disease, Heart Failure, and All-Cause mortality by C24:0/C16:0 Ceramide Ratio. Hazard ratios for coronary heart disease (CHD), heart failure (HF), and all-cause mortality are reported with 95% confidence intervals (CI) for each standard deviation increase C24:0/C16:0 ceramide ratio. Data is shown from analysis of subjects in FHS, SHIP and the combined meta-analysis.

FIG. 8 shows risk of Coronary Heart Disease, Heart Failure, and All-Cause mortality by C22:0/C16:0 Ceramide Ratio. Hazard ratios for coronary heart disease (CHD), heart failure (HF), and all-cause mortality are reported with 95% confidence intervals (CI) for each standard deviation increase in C22:0/C16:0 ceramide ratio. Data is shown from analysis of subjects in FHS, SHIP and the combined meta-analysis.

FIG. 9 shows risk of Cardiovascular Disease (CVD) Mortality and Non-Cardiovascular Disease (CVD) Mortality by Ceramide Ratios. Hazard ratios for cardiovascular disease (CVD) mortality and non-CVD mortality are reported with 95% confidence intervals (CI) for each standard deviation increase in C24:0/C16:0 or C22:0/C16:0 ceramide ratio. Data is shown from analysis of subjects in FHS, SHIP and the combined meta-analysis.

FIG. 10 shows risk of Coronary Heart Disease, Heart Failure, and All-Cause Mortality by C24:0 Ceramide Level. Hazard ratios for coronary heart disease (CHD), heart failure (HF), and all-cause mortality are reported with 95% confidence intervals (CI) for each standard deviation increase in C24:0 ceramide level. Data is shown from analysis of subjects in FHS, SHIP and the combined meta-analysis.

FIG. 11 shows risk of Coronary Heart Disease, Heart Failure, and All-Cause Mortality by C16:0 Ceramide Level. Hazard ratios for coronary heart disease (CHD), heart failure (HF), and all-cause mortality are reported with 95% confidence intervals (CI) for each standard deviation increase in C16:0 ceramide level. Data is shown from analysis of subjects in FHS, SHIP and the combined meta-analysis.

Those of skill in the art will understand that the drawings, described above, are for illustrative purposes only. The drawings are not intended to limit the scope of the present teachings in any way.

### DETAILED DESCRIPTION OF THE INVENTION

The present disclosure provides a statistically significant relationship between reduced levels of ratios of very long chain to long chain ceramides that indicates increased risk of cardiovascular related diseases, disorder, and conditions, increased risk of death from pancreatic cancer or all-cause mortality. In particular, it was discovered that a higher C24:0/C16:0 ceramide ratio was inversely associated with cardiovascular mortality. Moreover, the C24:0/C16:0 ceramide ratio was also inversely associated with non-cardiovascular mortality and all-cause mortality. Similar observations were observed for C22:0/C16:0 ceramide ratio. Additionally, C24:0/16:0 ceramide ratios were inversely associated with coronary heart disease. Thus, lower ratios of very long chain to long chain ceramides are associated with a greater incidence of coronary heart disease and greater incidence of cardiovascular, non-cardiovascular, and all-cause mortality.

It was unexpected the C24:0/C16:0 ceramide ratio and the C22:0/C16:0 ceramide ratio is inversely associated with the coronary risk factors. These results are opposite of the effects observed in previous studies (Haus et al., 2009; de Mello et al., 2009). Prior observations indicated that total plasma ceramides are directly associated with CVD risk factors suggesting that plasma ceramides reflect changes in lipid metabolism that promote CHD and mortality (Boon et al. 2013 Diabetes 62 401-10; de Mello et al. 2009 Diabetologia 52 2612-5; Haus et al. 2009 Diabetes 58 337-43). Instead, the present disclosure provides the C24:0/C16:0 ceramide ratio are inversely associated with the coronary risk factors of age and smoking status and also inversely associated with prevalent CVD. Accordingly, provided herein are methods that utilize this relationship to provide inventive means of assessing the risk of progression to CVD including related diseases, disorders, and conditions, and increased risk of death from pancreatic cancer or all-cause mortality, and inventive means of treating a subject based on risk of progression to CVD in order to modify the subject's risk and/or prevent CVD.

As described herein, sphingolipids (e.g., ceramides) have been implicated as prognostic indicators of cardiovascular disease, heart failure, cardiovascular death, or non-cardiovascular death. Ceramides are a family of waxy lipid molecules. Ceramides consist of a long-chain or sphingoid base linked to a fatty acid via an amide bond. Ceramides are found in high concentrations within the cell membrane of cells, since they are component lipids that make up sphingomyelin, one of the major lipids in the lipid bilayer. Contrary to previous assumptions that ceramides and other sphingolipids found in cell membrane were purely supporting structural elements, ceramide can participate in a variety of cellular signaling: examples include regulating differentiation, proliferation, or programmed cell death (PCD) of cells. Ceramides are known to be key players in intracellular signaling and are involved in, inter alia, apoptosis, cell senescence, proliferation, cell growth, and differentiation. Ceramides can be synthesized by ceramide synthases (CerS). It is currently believed that six different mammalian CerS (CerS1-6) have been described. As described herein, it was demonstrated ceramides have been shown to be prognostic indicators of cardiovascular disease, heart failure, cardiovascular death, or non-cardiovascular death. Specifically, the ceramide species include two very long chain species: C22:0 ceramide and C24:0 ceramide and one long-chain species: C16:0. C22:0 ceramide may also be referred to as d18:1/22:0, C24:0 ceramide may also be referred to as d18:1/24:0, and C16:0 may also be referred to as d18:1/C16:0.

Provided herein are methods for measuring C22:0 and C16:0 or C24:0 and C16:0 ceramide using a novel tandem MS assay and their use in classifying a subject at risk for CVD. As shown herein, liquid chromatography/mass spectrometry was used to quantify plasma d18:1/C24:0, d18:1/C22:0, and d18:1/C16:0 ceramides in 2,642 Framingham Heart Study (FHS) participants and in 3,135 Study of Health in Pomerania (SHIP) participants. All three ceramide species can be simultaneously quantified in ˜50 microliters of plasma using the novel FDA-compliant tandem mass spectrometry assay described herein.

As shown herein, liquid chromatography/mass spectrometry was used to quantify plasma d18:1/C24:0, d18:1/C22:0, and d18:1/C16:0 ceramides in 2,642 Framingham Heart Study (FHS) participants and in 3,135 Study of Health in Pomerania (SHIP) participants. All three ceramide species can be simultaneously quantified in ˜50 microliters of plasma using the novel FDA-compliant tandem mass spectrometry assay described herein.

One aspect of the present disclosure shows plasma ceramides were related to incidence of cardiovascular disease and all-cause mortality. It was discovered that over a mean follow up of 6 years in FHS, higher C24:0/C16:0 ceramide ratios were inversely associated with cardiovascular mortality (hazard ratio per standard deviation increment [HR] 0.55, 95% confidence interval (CI) [0.38, 0.79], P=0.0012). Moreover, it was discovered that the C24:0/C16:0 ceramide ratio was also inversely associated with non-cardiovascular mortality (HR 0.6, 95% CI [0.51, 0.70]. p<0.0001) and all-cause mortality (HR 0.6, 95% CI [0.50,0.71], p<0.0001). Similar associations were observed for C22:0/C16:0 ceramide ratio. These measures increased the C-statistic when added to standard coronary risk factors, indicating that they are new, independent risk factors. Plasma ceramides were also related to incidence of coronary heart disease. In meta-analyses of the FHS and SHIP cohorts, over a mean follow up of 6 years, C24:0/C16:0 ceramide ratios were inversely associated with coronary heart disease (HR=0.80, 95% CI [0.71, 0.91], P=0.0006). Findings for both C24:0/C16:0 and C22:0/C16:0 ratios were replicated in data from SHIP and in meta-analyses of the two studies.

Thus, in large community-based samples, lower ratios of very long chain to long chain ceramides were found to be associated with a greater incidence of coronary heart disease and greater incidence of cardiovascular, non-cardiovascular, and all-cause mortality. A simple blood test measuring these analytes, akin to the widely used fasting lipid profile, can be incorporated into standard clinical care to assess risk of both cardiovascular and non-cardiovascular death and represent potential targets for therapeutic intervention.

The present disclosure demonstrates the unexpected result that individuals with a reduced ceramide ratio have a 40% increased risk of death from cardiovascular disease and all causes over a 5-year period.

As described herein, the present disclosure used liquid chromatography/mass spectrometry to quantify plasma C24:0, C22:0, and C16:0 ceramides in 2,642 Framingham Heart Study (FHS) participants and in 3,135 Study of Health in Pomerania (SHIP) participants. Plasma ceramides were related to incidence of cardiovascular disease and all-cause mortality. Over a mean follow up of 5 years in FHS, higher C24:0/C16:0 ceramide ratios were inversely associated with cardiovascular mortality (hazard ratio per standard deviation increment [HR] 0.55, 95% CI [0.38, 0.79], P=0.0012). Moreover, the C24:0/C16:0 ceramide ratio was also inversely associated with non-cardiovascular mortality (HR 0.6, 95% CI [0.51,0.70], p<0.0001) and all-cause mortality (HR 0.6, 95% CI [0.51, 0.70], p<0.0001). Plasma ceramides were related to incidence of coronary heart disease. In meta-analyses of the FHS and SHIP cohorts, over a mean follow up of 6 years, C24:0/C16:0 ceramide ratios were inversely associated with coronary heart disease (HR=0.80, 95% CI [0.71, 0.91], P=0.0006). Similar associations were observed for C22:0/C16:0 ceramide ratio. As such, the presently disclosed novel assay can determine risk of mortality (e.g., from cardiovascular disease) by measuring the ratios of certain ceramides (C24:0 or C22:0 to C16:0) in plasma using the novel tandem mass spectrometry assay and the assay has been shown to predict the risk of death from cardiovascular disease (HR 0.55, CI 95%, p=0.0012), all-cause mortality (HR 0.6, CI 95%, p=0.0001), and coronary heart disease (HR 0.8, CI 95%, p=0.0006) 6 years in advance. Findings for both C24:0/C16:0 and C22:0/C16:0 ratios were replicated in data from SHIP and in meta-analyses of the two studies. In large community-based samples, higher plasma very long chain ceramides and lower ratios of very long chain to long chain ceramides are associated with a greater incidence of cardiovascular mortality and all-cause mortality. In addition, these measures increased the C-statistic when added to standard coronary risk factors (smoking, HDL/cholesterol, diabetes, etc.), indicating that these markers provide new information. The ratio is an improvement on the previously disclosed ceramide biomarker technology with greater predictive value.

Applications for the present disclosure can include use by insurance companies, use in an assay kit, use by companies that provide analytical instruments, and clinical reference laboratories. Additional applications include use by clinicians when treating subjects at risk of cardiovascular disease.

Various aspects of these methods are described in more detail below.

### I. Methods

In an aspect, the disclosure provides a method to classify a subject based on the amount of C16:0 and C22:0 or C16:0 and C24:0, measured in a biological sample obtained from the subject. The method generally comprises (i) measuring the amount of C16:0 and C22:0 or C16:0 and C24:0, in the biological sample, (ii) determining if a ratio of C22:0 to C16:0 or a ratio of C24:0 to C16:0 is increased or decreased relative to a reference value, and (iii) classifying the subject as having an increased or decreased risk of death or developing a cardiovascular disease, disorder, or condition. In some embodiments, the subject is classified as having an increased risk of death or developing a cardiovascular disease, disorder, or condition if the ratio of C22:0 to C16:0 or C24:0 to C16:0 is decreased relative to the reference value. In some embodiments, the subject is classified as having a decreased risk of death or developing a cardiovascular disease, disorder, or condition if the ratio of C22:0 to C16:0 or C24:0 to C16:0 is increased relative to the reference value.

In another aspect, the disclosure provides a method to determine if a subject is at risk of cardiovascular disease (CVD). The method generally comprises (i) measuring the amount of C16:0 and C22:0 or C16:0 and C24:0, in the biological sample, (ii) determining if a ratio of C22:0 to C16:0 or a ratio of C24:0 to C16:0 is increased or decreased relative to a reference value, and (iii) classifying the subject as having an increased risk of cardiovascular disease if the ratio of C22:0 to C16:0 or C24:0 to C16:0 is decreased relative to the reference value. The subject may have no other risk factors for CVD. Alternatively, the subject may have one or more risk factors for CVD. Non-limiting examples of risk factors for CVD include family history, ethnicity, age, sex, body mass index (BMI), tobacco exposure, high blood pressure (hypertension), high cholesterol, obesity, physical inactivity, diabetes, unhealthy diet, harmful use of alcohol, poverty, stress, social isolation, anxiety, depression, use of contraceptive pill, use of hormone replacement therapy, prior CVD, and left ventricular hypertrophy (LVH). Specifically, the CVD is coronary heart disease (CHD) or heart failure (HF). Additionally, the CVD includes fatal and non-fatal CHD, cerebrovascular disease (stroke or transient ischemic attack), peripheral arterial disease (intermittent claudication) and heart failure. CHD includes myocardial infarction (MI), coronary insufficiency and angina pectoris.

In still another aspect, the disclosure provides a method to prevent cardiovascular disease (CVD) in a subject. The method generally comprises (i) measuring the amount level of C16:0 and C22:0 or C16:0 and C24:0, in the biological sample, (ii) determining if a ratio of C22:0 to C16:0 or a ratio of C24:0 to C16:0 is increased or decreased relative to a reference value, and (iii) classifying the subject as having an increased risk of cardiovascular disease if the ratio of C22:0 to C16:0 or C24:0 to C16:0 is decreased relative to the reference value, and (iv) treating the subject to prevent future CVD events. The subject may have no history of CVD. Alternatively, the subject may have a history of CVD. Specifically, the CVD is coronary heart disease (CHD) or heart failure (HF). Additionally, the CVD includes fatal and non-fatal CHD, cerebrovascular disease (stroke or transient ischemic attack), peripheral arterial disease (intermittent claudication) and heart failure. CHD includes myocardial infarction (MI), coronary insufficiency and angina pectoris. Treatment may consist of standard treatments for CVD. Non-limiting examples of standard treatment for CVD include stress reduction, diet changes, lifestyle changes, drugs and surgery. Non-limiting examples of lifestyle changes include cessation of smoking, exercising, alcohol in moderation, and relaxation techniques such as mediation, progressive relaxation, yoga and biofeedback training. Non-limiting examples of diet changes include lowering sodium and trans fat consumption and increasing intake of fresh fruits and vegetables, whole unprocessed high-fiber grains, and healthy sources of fats and proteins. Non-limiting examples of drugs include aspirin, ACE inhibitors, angiotensin II receptor blockers, anti-arrhythmics, beta-blockers, high blood pressure medication, high cholesterol medication, diuretics, water pills, calcium channel blocker drugs, thrombolytic drugs, digoxin, nitrates, hydralazine, antiplatelet drugs, blood thinners, and corticosteroids. Non-limiting examples of percutaneous interventions and surgery include balloon angioplasty and stents, balloon valvuloplasty, heart bypass surgery, open heart surgery, pacemaker or defibrillator implantation, heart transplantation, cardioconversion, atrial fibrillation and bypass tract ablation and left ventricular assist device (LVAD).

In still yet another aspect, the disclosure provides a method for monitoring cardiovascular disease (CVD) in a subject. In such an embodiment, a method of detecting the amount of C16:0 and C22:0 or C16:0 and C24:0 and determining a ratio of C22:0 to C16:0 or a ratio of C24:0 to C16:0, may be used to assess the risk of a subject at one point in time. Then at a later time, the method of detecting the amount of C16:0 and C22:0 or C16:0 and C24:0, and determining a ratio of C22:0 to C16:0 or a ratio of C24:0 to C16:0, may be used to determine the change in risk of the subject over time. For example, the method of detecting the amount of C16:0 and C22:0 or C16:0 and C24:0, and determining a ratio of C22:0 to C16:0 or a ratio of C24:0 to C16:0, may be used on the same subject days, weeks, months or years following the initial determination of the amount of C16:0 and C22:0 or C16:0 and C24:0, and determining a ratio of C22:0 to C16:0 or a ratio of C24:0 to C16:0. Accordingly, the method of detecting the amount of C16:0 and C22:0 or C16:0 and C24:0, and determining a ratio of C22:0 to C16:0 or a ratio of C24:0 to C16:0, may be used to follow a subject over time to determine when the risk of progressing to more severe disease is high thereby requiring treatment. Additionally, the method of detecting the amount of C16:0 and C22:0 or C16:0 and C24:0, and determining a ratio of C22:0 to C16:0 or a ratio of C24:0 to C16:0, may be used to measure the rate of disease progression. For example, if the ratio of C22:0 to C16:0 or C24:0 to C16:0 is increased relative to the ratio of C22:0 to C16:0 or C24:0 to C16:0 obtained from the same subject at an earlier time point, may indicate an abatement of disease progression. Alternatively, if the ratio of C22:0 to C16:0 or C24:0 to C16:0 is decreased relative to the ratio of C22:0 to C16:0 or C24:0 to C16:0 obtained from the same subject at an earlier time point, may indicate disease progression.

In still yet another aspect, the disclosure provides a method of treating a cardiovascular disease (CVD) in a subject. In such an embodiment, a method of detecting the amount of C16:0 and C22:0 or C16:0 and C24:0 and determining a ratio of C22:0 to C16:0 or a ratio of C24:0 to C16:0, may be used to assess the risk of a subject at one point in time. Then at a later time, the method of detecting the amount of C16:0 and C22:0 or C16:0 and C24:0, and determining a ratio of C22:0 to C16:0 or a ratio of C24:0 to C16:0, may be used to determine the change in risk of the subject over time. For example, the method of detecting the amount of C16:0 and C22:0 or C16:0 and C24:0, and determining a ratio of C22:0 to C16:0 or a ratio of C24:0 to C16:0, may be used on the same subject days, weeks, months or years following the initial determination of the amount of C16:0 and C22:0 or C16:0 and C24:0, and determining a ratio of C22:0 to C16:0 or a ratio of C24:0 to C16:0. Accordingly, the method of detecting the amount of C16:0 and C22:0 or C16:0 and C24:0, and determining a ratio of C22:0 to C16:0 or a ratio of C24:0 to C16:0, may be used to follow a subject over time to determine when the risk of progressing to more severe disease is high thereby requiring treatment.

Additionally, a method for monitoring cardiovascular disease (CVD) in a subject may also be used to determine the response to treatment. As used herein, subjects who respond to treatment are said to have benefited from treatment. Responses to treatment are measured in clinical practice using tests including, but not limited to, blood pressure test, LDL cholesterol test, chest X-ray, electrocardiogram (ECG), Holter monitoring, echocardiogram, stress test, blood test, cardiac catheterization, electrophysiology test, CT heart scan, myocardial biopsy, heart MRI, and pericardiocentesis. These tests are well known in the art and are intended to refer to specific parameters measured during clinical trials and in clinical practice by a skilled artisan. For example, a method of detecting the amount of C16:0 and C22:0 or C16:0 and C24:0, and determining a ratio of C22:0 to C16:0 or a ratio of C24:0 to C16:0, may be performed on the biological sample of the subject prior to initiation of treatment. Then at a later time, a method of detecting the amount of C16:0 and C22:0 or C16:0 and C24:0, and determining a ratio of C22:0 to C16:0 or a ratio of C24:0 to C16:0, may be used to determine the response to treatment over time. For example, a method of detecting the amount of C16:0 and C22:0 or C16:0 and C24:0, and determining a ratio of C22:0 to C16:0 or a ratio of C24:0 to C16:0, may be performed on the biological sample of the same subject days, weeks, months or years following initiation of treatment. Accordingly, a method of detecting the amount of C16:0 and C22:0 or C16:0 and C24:0, and determining a ratio of C22:0 to C16:0 or a ratio of C24:0 to C16:0, may be used to follow a subject receiving treatment to determine if the subject is responding to treatment. If the ratio of C22:0 to C16:0 or C24:0 to C16:0 is increased, then the subject may be responding to treatment. If the ratio of C22:0 to C16:0 or C24:0 to C16:0 decreases or remains the same, then the subject may not be responding to treatment. These steps may be repeated to determine the response to therapy over time.

In one aspect, the disclosure provides a method of treating a cardiovascular disease (CVD), disorders or conditions in a subject in need thereof. As used herein, subjects who respond to treatment are said to have benefited from treatment. Responses to treatment are measured in clinical practice using tests including, but not limited to, blood pressure test, LDL cholesterol test, chest X-ray, electrocardiogram (ECG), Holter monitoring, echocardiogram, stress test, blood test, cardiac catheterization, electrophysiology test, CT heart scan, myocardial biopsy, heart MRI, and pericardiocentesis. These tests are well known in the art and are intended to refer to specific parameters measured during clinical trials and in clinical practice by a skilled artisan. For example, a method of detecting the amount of C16:0 and C22:0 or C16:0 and C24:0, and determining a ratio of C22:0 to C16:0 or a ratio of C24:0 to C16:0, may be performed on the biological sample of the subject prior to initiation of treatment. Then at a later time, a method of detecting the amount of C16:0 and C22:0 or C16:0 and C24:0, and determining a ratio of C22:0 to C16:0 or a ratio of C24:0 to C16:0, may be used to determine the response to treatment over time. For example, a method of detecting the amount of C16:0 and C22:0 or C16:0 and C24:0, and determining a ratio of C22:0 to C16:0 or a ratio of C24:0 to C16:0, may be performed on the biological sample of the same subject days, weeks, months or years following initiation of treatment. Accordingly, a method of detecting the amount of C16:0 and C22:0 or C16:0 and C24:0, and determining a ratio of C22:0 to C16:0 or a ratio of C24:0 to C16:0, may be used to follow a subject receiving treatment to determine if the subject is responding to treatment. If the ratio of C22:0 to C16:0 or C24:0 to C16:0 is increased, then the subject may be responding to treatment. If the ratio of C22:0 to C16:0 or C24:0 to C16:0 decreases or remains the same, then the subject may not be responding to treatment. These steps may be repeated to determine the response to therapy over time.

Cardiovascular disease is the major cause of mortality in the developed world. Effective primary prevention of cardiovascular disease requires identification of significant risk factors, followed by intervention to modify risk. This strategy has been very effective with respect to hypercholesterolemia, which can be detected by a blood test—a fasting lipid profile—and drug therapy with a cholesterol-lowering agent, such as a statin. The plasma very long chain ceramide species identified in the present studies represent powerful independent risk factors for cardiovascular disease that are additive to traditional risk factors. Since these ceramide species can be accurately and readily quantified in plasma of human subjects using the presently disclosed validated tandem mass spectrometry assay, there is little barrier to widespread adoption of this blood test for cardiovascular disease risk stratification in the US population, similar to standard blood cholesterol measurements. Identification of drugs that alter plasma ceramide levels and demonstration that this intervention mitigates cardiovascular and all-cause mortality would provide further rationale for application of this plasma marker.

According to the present disclosure, a method of detecting the amount of C16:0 and C22:0 or C16:0 and C24:0, and determining a ratio of C22:0 to C16:0 or a ratio of C24:0 to C16:0, may indicate an increased risk of developing cardiovascular-related diseases, disorders, or conditions, and increased risk of cardiac-related or all-cause death, for example, within 6 years. For example, ratios of very long to long chain ceramide ratios may be associated with increased risk of developing cardiovascular-related diseases, disorders, or conditions, and increased risk of cardiac-related or all-cause death within 20 years or less, 19 years or less, 18 years or less, 17 years or less, 16 years or less, 15 years or less, 14 years or less, 13 years or less, 12 years or less, 11 years or less, 10 years or less, 9 years or less, 8 years or less, 7 years or less, 6 years or less, 5 years or less, 4 years or less, 3 years or less, 2 years or less, or 1 year or less.

Biomarkers as described herein can be a ceramide (Cer). A ceramide can be of the following formula, where R represents the alkyl portion of the fatty acid.

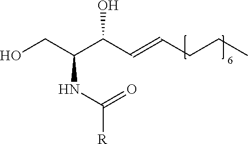

For example, a Cer can be any ceramide known in the art such as a short chain ceramide, a long chain ceramide, or a very long chain ceramide. A short chain ceramide can be a C4-C12 ceramide. A longer chain ceramide can be a C12-C18 ceramide or a C16-C24 ceramide. A long chain ceramides can be a C16-C24 ceramide. A very long chain ceramide can include a C22-C24 ceramide. For example, a ceramide can be a C4, C5, C6, C7, C8, C9, C10, C12, C13, C14, C15, C16, C17, C18, C19, C20, C21, C22, C23, C24, C25, or C26 ceramide. As another example, a ceramide can be a C4:0, C5:0, C6:0, C7:0, C8:0, C9:0, C10:0, C12:0, C13:0, C14:0, C15:0, C16:0, C17:0, C18:0, C19:0, C20:0, C21:0, C22:0, C23:0, C24:0, C25:0, or C26:0 ceramide. As another example, a ceramide can be a C4:1, C5:1, C6:1, C7:1, C8:1, C9:1, C10:1, C12:1, C13:1, C14:1, C15:1, C16:1, C17:1, C18:1, C19:1, C20:1, C21:1, C22:1, C23:1, C24:1, C25:1, or C26:1 ceramide. As another example, the ceramide can be d18:0, d18:1, d18:2, d17:0, or d17:1.

According to conventional lipid nomenclature for ceramides, annotation of sphingoid backbone denotes number of hydroxyl group, number of carbon and number of unsaturation degree (e.g., m denotes one hydroxyl group; d denotes two hydroxyl groups; t denotes three hydroxyl groups); annotation of N-acyl chain indicates number of carbon, number of unsaturation degree and number of hydroxyl group (e.g., in d18:1/24:0(OH), 24 denotes number of total carbon; 0 denotes number of unsaturation degree; OH denotes one hydroxyl group). For some ceramides with polyhydroxyl N-acyl chain, dOH and tOH denotes two hydroxyl groups and three hydroxyl groups in N-acyl chain, respectively. For example, Cer (t18:1/42:1(dOH)) and Cer (t14:1/22:1(tOH)).

As described herein, liquid chromatography/mass spectrometry may be used to quantify plasma ceramides. For example, the plasma ceramides detected can be d18:1/C24:0, d18:1/C22:0, or d18:1/C16:0 ceramides.

High levels of total tissue ceramides have been implicated in both metabolic and cardiovascular diseases in animal models. In rodents, genetic and pharmacological interventions that decrease total plasma ceramides prevent diet- and glucocorticoid-induced insulin resistance and diabetes, thus implicating ceramides as key drivers of metabolic diseases. In murine models of atherosclerosis and metabolic cardiomyopathy, broad inhibition of de novo ceramide synthesis prevents lesion formation and cardiac structural and functional abnormalities. These studies raise the possibility that ceramides could serve as a useful diagnostic tool and/or serve as therapeutic targets.

Nonetheless, conflicting findings have been reported from studies of small cohorts of human subjects that have related circulating ceramides to outcomes. In some individuals with obesity and type 2 diabetes, higher total ceramide levels in plasma, skeletal muscle, liver and adipose tissue correlate positively with insulin resistance and inflammation, whereas in other studies, no differences were found in skeletal muscle ceramide content between subjects with type 2 diabetes and controls. Total plasma and cardiac tissue ceramides were elevated in subjects with hypertension, coronary heart disease (CHD) and heart failure (HF), but not in cardiac tissue of patients who are obese and/or have type 2 diabetes. Interventions that improve insulin resistance have been associated with variable effects on total plasma ceramide levels.

Recent results raise the possibility that the deleterious cardiometabolic effects of ceramides may relate to remodeling of the ceramide acyl chain species, rather than total ceramide levels. In mice with altered expression of ceramide synthase 2 and ceramide synthase 6, increases in long chain ceramide species (e.g., C16:0 ceramide) and decreases in very long chain species (e.g., C22:0 and C24:0 ceramides) in metabolic tissues are associated with diet-induced glucose intolerance, non-alcoholic steatohepatitis, and insulin resistance. Cross-sectional analyses of gene expression and ceramide content of visceral white adipose tissue from human subjects demonstrate that increases in ceramide synthase 6 expression and long chain ceramides correlate positively with body mass index (BMI). In retrospective case-control studies of patients with CHD, high plasma C16:0 ceramide, low C24:0 ceramide, and low C24:0 to C16:0 ceramide ratios were directly related to cardiovascular mortality over three years.

The present disclosure provides results to answer whether changes in circulating ceramide acyl chain distribution are associated with risk of CHD or mortality in an unselected sample of the general population, which was previously unknown. To address this question, the present disclosure provides for relating levels of the most abundant long and very long chain ceramides in plasma to the risk of CHD and all-cause mortality in two large community-based samples.

Decreased levels of very long ceramides to long ceramide ratios can indicate an increased risk of a subject to subject be at an increased risk of developing cardiovascular disease (CVD), cardiovascular mortality, non-cardiovascular mortality, developing cardiac heart disease, suffering from heart failure, CVD mortality, or all-cause mortality. For example, the increased risk over a period of time as described herein can be about 40% or more. As another example, the increased risk over a period of time as described herein, can be or can be an increase in risk of about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; about 100%; about 101%; about 102%; about 103%; about 104%; about 105%; about 106%; about 107%; about 108%; about 109%; about 110%; about 111%; about 112%; about 113%; about 114%; about 115%; about 116%; about 117%; about 118%; about 119%; about 120%; about 121%; about 122%; about 123%; about 124%; about 125%; about 126%; about 127%; about 128%; about 129%; about 130%; about 131%; about 132%; about 133%; about 134%; about 135%; about 136%; about 137%; about 138%; about 139%; about 140%; about 141%; about 142%; about 143%; about 144%; about 145%; about 146%; about 147%; about 148%; about 149%; about 150%; about 151%; about 152%; about 153%; about 154%; about 155%; about 156%; about 157%; about 158%; about 159%; about 160%; about 161%; about 162%; about 163%; about 164%; about 165%; about 166%; about 167%; about 168%; about 169%; about 170%; about 171%; about 172%; about 173%; about 174%; about 175%; about 176%; about 177%; about 178%; about 179%; about 180%; about 181%; about 182%; about 183%; about 184%; about 185%; about 186%; about 187%; about 188%; about 189%; about 190%; about 191%; about 192%; about 193%; about 194%; about 195%; about 196%; about 197%; about 198%; about 199%; or about 200%. Recitation of each of these discrete values is understood to include ranges between each value.

By “cardiovascular disease” is meant a class of diseases that involve the heart or blood vessels. CVD includes, but is not limited to, coronary artery diseases (CAD) [also known as coronary heart disease (CHD) and ischemic heart disease] such as angina pectoris, coronary insufficiency, and myocardial infarction (heart attack), peripheral arterial disease (intermittent claudication), cerebrovascular disease (such as stroke or transient ischemic attack), renal artery stenosis, aortic aneurysm, cardiomyopathy, hypertensive heart disease, heart failure, pulmonary heart disease, cardiac dysrhythmias (such as atrial fibrillation), inflammatory heart disease (such as endocarditis, inflammatory cardiomegaly, and myocarditis), valvular heart disease, rheumatic heart disease, congenital heart disease, and venous thrombosis.

Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In preferred embodiments, the animal is a laboratory animal. Non-limiting examples of a laboratory animal may include rodents, canines, felines, and non-human primates. In certain embodiments, the animal is a rodent. In a preferred embodiment, the subject is human.

A subject may or may not be having a symptom associated with CVD. Specifically, the CVD may be coronary heart disease (CHD) or heart failure (HF). Additionally, the CVD includes fatal and non-fatal CHD, cerebrovascular disease (stroke or transient ischemic attach), peripheral arterial disease (intermittent claudication) and heart failure. CHD includes myocardial infarction (MI), coronary insufficiency and angina pectoris. A skilled artisan will appreciate that pathological CVD likely commences prior to diagnosis or the onset of symptoms associated with CVD. In some embodiments, a subject is having a symptom associated with CVD. In other embodiments, a subject is not having a symptom associated with CVD. In still other embodiments, a subject has detectable CVD but is not having any other symptom associated with CVD. In yet still other embodiments, a subject has received treatment for CVD. Early assessment of the risk of CVD in the subject may reduce the development and/or progression of symptoms associated with the pathological CVD by enabling improved interventions or enabling earlier interventions.

Exemplary symptoms associated with CVD include, but are not limited to, angina or chest pain (discomfort, heaviness, pressure, aching, burning, fullness, squeezing, or painful feeling in your chest); shortness of breath; heart palpitations; faster heartbeat; weakness or dizziness; nausea; sweating; discomfort, pressure, heaviness, or pain in the arm or below the breastbone; discomfort radiating to the back, jaw, throat, or arm; fullness, indigestion or choking feeling; vomiting; anxiety; rapid or irregular heart; lack of energy; swelling of ankles or feet; swelling in abdomen; quick weight gain; cough that produces white sputum; and limited ability to exercise.

As used herein, the term “biological sample” refers to a sample obtained from a subject. Any biological sample containing ceramides is suitable. Numerous types of biological samples are known in the art. Suitable biological sample may include, but are not limited to, tissue samples or bodily fluids. In some embodiments, the biological sample is a tissue sample such as a tissue biopsy. The biopsied tissue may be fixed, embedded in paraffin or plastic, and sectioned, or the biopsied tissue may be frozen and cryosectioned. Alternatively, the biopsied tissue may be processed into individual cells or an explant, or processed into a homogenate, a cell extract, a membranous fraction, or a ceramide extract. In other embodiments, the sample may be a bodily fluid. Non-limiting examples of suitable bodily fluids include blood, plasma, serum, urine, and saliva. In a specific embodiment, the biological sample is blood, plasma, or serum. In a specific embodiment, the biological sample is plasma. The fluid may be used “as is”, the cellular components may be isolated from the fluid, or a ceramide fraction may be isolated from the fluid using standard techniques.

As will be appreciated by a skilled artisan, the method of collecting a biological sample can and will vary depending upon the nature of the biological sample and the type of analysis to be performed. Any of a variety of methods generally known in the art may be utilized to collect a biological sample. Generally speaking, the method preferably maintains the integrity of the sample such that the ceramides can be accurately detected and the amount measured according to the disclosure.

In some embodiments, a single sample is obtained from a subject to detect ceramide in the sample. Alternatively, ceramide may be detected in samples obtained over time from a subject. As such, more than one sample may be collected from a subject over time. For instance, 2, 3, 4, 5, 6, 7, 8, 9, 10, 11, 12, 13, 14, 15, 16 or more samples may be collected from a subject over time. In some embodiments, 2, 3, 4, 5, or 6 samples are collected from a subject over time. In other embodiments, 6, 7, 8, 9, or 10 samples are collected from a subject over time. In yet other embodiments, 10, 11, 12, 13, or 14 samples are collected from a subject over time. In other embodiments, 14, 15, 16 or more samples are collected from a subject over time.

When more than one sample is collected from a subject over time, samples may be collected every 0.5, 1, 2, 3, 4, 5, 6, 7, 8, 9, 10, 11, 12 or more hours. In some embodiments, samples are collected every 0.5, 1, 2, 3, or 4 hours. In other embodiments, samples are collected every 4, 5, 6, or 7 hours. In yet other embodiments, samples are collected every 7, 8, 9, or 10 hours. In other embodiments, samples are collected every 10, 11, 12 or more hours. Additionally, samples may be collected every 1, 2, 3, 4, 5, 6, 7, 8, 9, 10, 11, 12 or more days. In some embodiments, a sample is collected about every 6 days. In some embodiments, samples are collected every 1, 2, 3, 4, or 5 days. In other embodiments, samples are collected every 5, 6, 7, 8, or 9 days. In yet other embodiments, samples are collected every 9, 10, 11, 12 or more days.

In some embodiments, once a sample is obtained, it is processed in vitro to detect and measure the amount of ceramide. All suitable methods for detecting and measuring an amount of ceramide known to one of skill in the art are contemplated within the scope of the invention. For example, epitope binding agent assays (i.e. antibody assays), enzymatic assays, electrophoresis, chromatography and/or mass spectrometry may be used. Non-limiting examples of epitope binding agent assays include an ELISA, a lateral flow assay, a sandwich immunoassay, a radioimmunoassay, an immunoblot or Western blot, flow cytometry, immunohistochemistry, and an array. In one embodiment, ceramides are detected using mass spectrometry. Ceramides may be detected through direct infusion into the mass spectrometer. In another embodiment, ceramides are detected using chromatography. In particular, techniques linking a chromatographic step with a mass spectrometry step may be used. The chromatographic step may be liquid chromatography, gas chromatography or thin-layer chromatography (TLC). Generally speaking, the presence of ceramides may be determined utilizing liquid chromatography followed by mass spectrometry. In some embodiments, the liquid chromatography is high performance liquid chromatography (HPLC). Non-limiting examples of HPLC include partition chromatography, normal phase chromatography, displacement chromatography, reverse phase chromatography, size exclusion chromatography, ion exchange chromatography, bioaffinity chromatography, aqueous normal phase chromatography or ultrafast liquid chromatography. Non-limiting examples of mass spectrometry include constant neutral loss mass spectrometry, tandem mass spectrometry (MS/MS), matrix-assisted laser desorption/ionization (MALDI), electrospray ionization mass spectrometry (ESI-MS). Described herein is a novel FDA-compliant tandem mass spectrometry assay to detect long and very long chain ceramides (see e.g., Example 1). Other methods of quantifying sphingolipids can include multiple reaction monitoring (MRM), “infusion” techniques, nanospray ionization, or ultra-high resolution mass analysis. Infusion techniques can use the same precursor ion/neutral loss scans and can be useful for profiling, but not quantitation, and can suffer from ionization suppression, isotopic, isobaric, and isomeric interferences especially without hydrolysis and extraction. Nanospray ionization can have an improved sensitivity, reduction in chemical noise, and allows detection of low abundance species, detailed structural analyses on numerous species, chip-based systems can be coupled to LC, and fraction collection. Ultra high resolution mass analysis can allow for differentiation of isobaric/isotopic interferences and alternative fragmentation techniques. In a specific embodiment, the method for detecting and measuring the amount of ceramide in a biological sample is liquid chromatography followed by tandem mass spectrometry (LC-MS/MS). More specifically, the method for detecting and measuring the amount of ceramide in a biological sample is the novel FDA-compliant tandem mass spectrometry assay described herein.

Any suitable reference value known in the art may be used. For example, a suitable reference value may be the amount of ceramide in a biological sample obtained from a subject or group of subjects of the same species that has no detectable CVD. In another example, a suitable reference value may be the amount of ceramide in a biological sample obtained from a subject or group of subjects of the same species that has detectable CVD as measured via standard methods. In another example, a suitable reference value may be a measurement of the amount of ceramide in a reference sample obtained from the same subject. The reference sample comprises the same type of biological fluid as the test sample, and may or may not be obtained from the subject when CVD was not suspected. A skilled artisan will appreciate that it is not always possible or desirable to obtain a reference sample from a subject when the subject is otherwise healthy. For example, in an acute setting, a reference sample may be the first sample obtained from the subject at presentation. In another example, when monitoring the effectiveness of a therapy, a reference sample may be a sample obtained from a subject before therapy began. In such an example, a subject may have suspected CVD but may not have other symptoms of CVD or the subject may have suspected CVD and one or more other symptom of CVD. In a specific embodiment, a suitable reference value may be a threshold provided in the Examples.

The term “risk” as used herein refers to the probability that an event will occur over a specific time period. For example, the probability that a CVD event will occur within 6, 12, 18, or 24 months or 3, 4 or 5 years after testing. Risk can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

Decreased levels of very long ceramides to long ceramide ratios may indicate about a 5% to about a 100% increased risk of developing CVD over a period of months or years. For example, a decreased ratio of ceramide 24:0 to ceramide 16:0 or decreased ratio of ceramide 22:0 to ceramide 16:0 relative to a reference value may indicate about a 5% to about a 95%, about a 5% to about a 90%, about a 5% to about a 85%, about a 5% to about a 80%, about a 5% to about a 75%, about a 5% to about a 70%, about a 5% to about a 65%, about a 5% to about a 60%, about a 5% to about a 55%, about a 5% to about a 50%, about a 5% to about a 45%, about a 5% to about a 40%, or about a 5% to about a 35% increased risk for developing CVD. Specifically, every standard deviation decrease in the ratio of ceramide 24:0 to ceramide 16:0 or ceramide 22:0 to ceramide 16:0 relative to a reference value may correspond to about a 5% to about a 50% increased risk of developing CVD. For example, every standard deviation decrease in of very long ceramides to long ceramide ratios relative to a reference value may correspond to about a 5% to about a 50%, about a 5% to about a 45%, about a 5% to about a 40%, about a 5% to about a 35%, about a 10% to about a 50%, about a 10% to about a 45%, about a 10% to about a 40%, about a 10% to about a 35%, about a 15% to about a 50%, about a 15% to about a 45%, about a 15% to about a 40%, or about a 15% to about a 35% increased risk of developing CVD.

The determination of risk may be used to select treatment for CVD subjects. As explained herein, decreased very long ceramides to long ceramide ratios, can classify a subject as having an increased risk of CVD and into groups that might benefit from therapy. In an embodiment, a subject classified as having an increased risk of CVD may be treated. A skilled artisan would be able to determine standard treatment for CVD. Accordingly, the methods disclosed herein may be used to select treatment for CVD subjects. In an embodiment, the subject is treated based on the difference in amount of ceramides relative to the reference level. This classification may be used to identify groups that are in need of treatment or not or in need of more aggressive treatment. The term “treatment” or “therapy” as used herein means any treatment suitable for the treatment of CVD. Treatment may consist of standard treatments for CVD. Non-limiting examples of standard treatment for CVD include stress reduction, diet changes, lifestyle changes, drugs and surgery. Non-limiting examples of lifestyle changes include cessation of smoking, exercising, alcohol in moderation, and relaxation techniques such as mediation, progressive relaxation, yoga and biofeedback training. Non-limiting examples of diet changes include lowering sodium and trans fat consumption and increasing intake of fresh fruits and vegetables, whole unprocessed high-fiber grains, and healthy sources of fats and proteins. Non-limiting examples of drugs include aspirin, ACE inhibitors, angiotensin II receptor blockers, anti-arrhythmics, beta-blockers, high blood pressure medication, high cholesterol medication, diuretics, water pills, calcium channel blocker drugs, thrombolytic drugs, digoxin, nitrates, hydralazine, antiplatelet drugs, blood thinners, and corticosteroids. Non-limiting examples of percutaneous interventions and surgery include balloon angioplasty and stents, balloon valvuloplasty, heart bypass surgery, open heart surgery, pacemaker or defibrillator implantation, heart transplantation, cardioconversion, atrial fibrillation and bypass tract ablation and left ventricular assist device (LVAD).

As described herein, the present disclosure provides for new methods to predict mortality from non-cardiovascular related death or predict all-cause mortality (including CVD related mortality). All-cause mortality can include any cause of death including cardiac related and non-cardiac related death (e.g., cancer death).

A control sample, as described herein can be a sample without any endogenous ceramide. A control sample can also be referred to as a calibration standard. For example, the control sample can be spiked with ceramides and optionally serially diluted. As another example, the control sample can be a BSA solution. A BSA solution with no ceramides can be a blank. The BSA solution can be of any concentration known in the art. For example, the solution can be about 0% BSA, about 1% BSA; about 2% BSA; about 3% BSA; about 4% BSA; about 5% BSA; about 6% BSA; about 7% BSA; about 8% BSA; about 9% BSA; about 10% BSA; about 11% BSA; about 12% BSA; about 13% BSA; about 14% BSA; about 15% BSA; about 16% BSA; about 17% BSA; about 18% BSA; about 19% BSA; about 20% BSA; about 21% BSA; about 22% BSA; about 23% BSA; about 24% BSA; about 25% BSA; about 26% BSA; about 27% BSA; about 28% BSA; about 29% BSA; about 30% BSA; about 31% BSA; about 32% BSA; about 33% BSA; about 34% BSA; about 35% BSA; about 36% BSA; about 37% BSA; about 38% BSA; about 39% BSA; about 40% BSA; about 41% BSA; about 42% BSA; about 43% BSA; about 44% BSA; about 45% BSA; about 46% BSA; about 47% BSA; about 48% BSA; about 49% BSA; about 50% BSA; about 51% BSA; about 52% BSA; about 53% BSA; about 54% BSA; about 55% BSA; about 56% BSA; about 57% BSA; about 58% BSA; about 59% BSA; about 60% BSA; about 61% BSA; about 62% BSA; about 63% BSA; about 64% BSA; about 65% BSA; about 66% BSA; about 67% BSA; about 68% BSA; about 69% BSA; about 70% BSA; about 71% BSA; about 72% BSA; about 73% BSA; about 74% BSA; about 75% BSA; about 76% BSA; about 77% BSA; about 78% BSA; about 79% BSA; about 80% BSA; about 81% BSA; about 82% BSA; about 83% BSA; about 84% BSA; about 85% BSA; about 86% BSA; about 87% BSA; about 88% BSA; about 89% BSA; about 90% BSA; about 91% BSA; about 92% BSA; about 93% BSA; about 94% BSA; about 95% BSA; about 96% BSA; about 97% BSA; about 98% BSA; about 99% BSA; or about 100% BSA. Recitation of each of these discrete values is understood to include ranges between each value.

A control sample can be spiked with any ceramide. For example, the control sample can be spiked with a C24:0, a C22:0, or a C16:0 ceramide.

A control sample can be serially diluted into any ratio for use as a control. For example, the ceramide can be at a concentration of about 0.01 μg/mL; about 0.02 μg/mL; about 0.03 μg/mL; about 0.04 μg/mL; about 0.05 μg/mL; about 0.06 μg/mL; about 0.07 μg/mL; about 0.08 μg/mL; about 0.09 μg/mL; about 0.1 μg/mL; about 0.2 μg/mL; about 0.3 μg/mL; about 0.4 μg/mL; about 0.5 μg/mL; about 0.6 μg/mL; about 0.7 μg/mL; about 0.8 μg/mL; about 0.9 μg/mL; about 1 μg/mL; about 1.5 μg/mL; about 2 μg/mL; about 2.5 μg/mL; about 3 μg/mL; about 3.5 μg/mL; about 4 μg/mL; about 4.5 μg/mL; about 5 μg/mL; about 5.5 μg/mL; about 6 μg/mL; about 6.5 μg/mL; about 7 μg/mL; about 7.5 μg/mL; about 8 μg/mL; about 8.5 μg/mL; about 9 μg/mL; about 9.5 μg/mL; about 10 μg/mL; about 10.5 μg/mL; about 11 μg/mL; about 11.5 μg/mL; about 12 μg/mL; about 12.5 μg/mL; about 13 μg/mL; about 13.5 μg/mL; about 14 μg/mL; about 14.5 μg/mL; about 15 μg/mL; about 15.5 μg/mL; about 16 μg/mL; about 16.5 μg/mL; about 17 μg/mL; about 17.5 μg/mL; about 18 μg/mL; about 18.5 μg/mL; about 19 μg/mL; about 19.5 μg/mL; about 20 μg/mL; about 20.5 μg/mL; about 21 μg/mL; about 21.5 μg/mL; about 22 μg/mL; about 22.5 μg/mL; about 23 μg/mL; about 23.5 μg/mL; about 24 μg/mL; about 24.5 μg/mL; about 25 μg/mL; about 25.5 μg/mL; about 26 μg/mL; about 26.5 μg/mL; about 27 μg/mL; about 27.5 μg/mL; about 28 μg/mL; about 28.5 μg/mL; about 29 μg/mL; about 29.5 μg/mL; about 30 μg/mL; about 30.5 μg/mL; about 31 μg/mL; about 31.5 μg/mL; about 32 μg/mL; about 32.5 μg/mL; about 33 μg/mL; about 33.5 μg/mL; about 34 μg/mL; about 34.5 μg/mL; about 35 μg/mL; about 35.5 μg/mL; about 36 μg/mL; about 36.5 μg/mL; about 37 μg/mL; about 37.5 μg/mL; about 38 μg/mL; about 38.5 μg/mL; about 39 μg/mL; about 39.5 μg/mL; about 40 μg/mL; about 40.5 μg/mL; about 41 μg/mL; about 41.5 μg/mL; about 42 μg/mL; about 42.5 μg/mL; about 43 μg/mL; about 43.5 μg/mL; about 44 μg/mL; about 44.5 μg/mL; about 45 μg/mL; about 45.5 μg/mL; about 46 μg/mL; about 46.5 μg/mL; about 47 μg/mL; about 47.5 μg/mL; about 48 μg/mL; about 48.5 μg/mL; about 49 μg/mL; about 49.5 μg/mL; or about 50 μg/mL. For example, the control sample can be serially diluted into 0.01/0.04/0.1, 0.02/0.08/0.2, 0.05/0.2/0.5, 0.1/0.4/1, 0.2/0.8/2, 0.5/2/5, 1/4/10, or 2/8/20 μg/mL of C16:0/C22:0/C24:0 ceramide solutions. Recitation of each of these discrete values is understood to include ranges between each value.

A control sample can be a human control sample such as a biological sample (e.g., plasma, blood). A human control sample can be from a healthy human subject. For example, the human subject can be, for example, non-smoking, aged 40-60 years old, free of hypertension, free of diabetes (hemoglobin A1c<6.5% and normal glucose tolerance test), free of cardiovascular disease (e.g., normal stress echocardiogram), or free of other major systemic illness. The biological sample can be drawn as a morning fasting blood draw.

A control sample can include one or more human control samples. For example, a control sample can be a statistically significant number of subjects. As another example, the control sample can comprise at least at least about 1 subject; at least about 2 subjects; at least about 3 subjects; at least about 4 subjects; at least about 5 subjects; at least about 6 subjects; at least about 7 subjects; at least about 8 subjects; at least about 9 subjects; at least about 10 subjects; at least about 11 subjects; at least about 12 subjects; at least about 13 subjects; at least about 14 subjects; at least about 15 subjects; at least about 16 subjects; at least about 17 subjects; at least about 18 subjects; at least about 19 subjects; at least about 20 subjects; at least about 21 subjects; at least about 22 subjects; at least about 23 subjects; at least about 24 subjects; at least about 25 subjects; at least about 26 subjects; at least about 27 subjects; at least about 28 subjects; at least about 29 subjects; at least about 30 subjects; at least about 31 subjects; at least about 32 subjects; at least about 33 subjects; at least about 34 subjects; at least about 35 subjects; at least about 36 subjects; at least about 37 subjects; at least about 38 subjects; at least about 39 subjects; at least about 40 subjects; at least about 41 subjects; at least about 42 subjects; at least about 43 subjects; at least about 44 subjects; at least about 45 subjects; at least about 46 subjects; at least about 47 subjects; at least about 48 subjects; at least about 49 subjects; at least about 50 subjects; at least about 51 subjects; at least about 52 subjects; at least about 53 subjects; at least about 54 subjects; at least about 55 subjects; at least about 56 subjects; at least about 57 subjects; at least about 58 subjects; at least about 59 subjects; at least about 60 subjects; at least about 61 subjects; at least about 62 subjects; at least about 63 subjects; at least about 64 subjects; at least about 65 subjects; at least about 66 subjects; at least about 67 subjects; at least about 68 subjects; at least about 69 subjects; at least about 70 subjects; at least about 71 subjects; at least about 72 subjects; at least about 73 subjects; at least about 74 subjects; at least about 75 subjects; at least about 76 subjects; at least about 77 subjects; at least about 78 subjects; at least about 79 subjects; at least about 80 subjects; at least about 81 subjects; at least about 82 subjects; at least about 83 subjects; at least about 84 subjects; at least about 85 subjects; at least about 86 subjects; at least about 87 subjects; at least about 88 subjects; at least about 89 subjects; at least about 90 subjects; at least about 91 subjects; at least about 92 subjects; at least about 93 subjects; at least about 94 subjects; at least about 95 subjects; at least about 96 subjects; at least about 97 subjects; at least about 98 subjects; at least about 99 subjects; at least about 100 subjects; at least about 101 subjects; at least about 102 subjects; at least about 103 subjects; at least about 104 subjects; at least about 105 subjects; at least about 106 subjects; at least about 107 subjects; at least about 108 subjects; at least about 109 subjects; at least about 110 subjects; at least about 111 subjects; at least about 112 subjects; at least about 113 subjects; at least about 114 subjects; at least about 115 subjects; at least about 116 subjects; at least about 117 subjects; at least about 118 subjects; at least about 119 subjects; at least about 120 subjects; at least about 121 subjects; at least about 122 subjects; at least about 123 subjects; at least about 124 subjects; at least about 125 subjects; at least about 126 subjects; at least about 127 subjects; at least about 128 subjects; at least about 129 subjects; at least about 130 subjects; at least about 131 subjects; at least about 132 subjects; at least about 133 subjects; at least about 134 subjects; at least about 135 subjects; at least about 136 subjects; at least about 137 subjects; at least about 138 subjects; at least about 139 subjects; at least about 140 subjects; at least about 141 subjects; at least about 142 subjects; at least about 143 subjects; at least about 144 subjects; at least about 145 subjects; at least about 146 subjects; at least about 147 subjects; at least about 148 subjects; at least about 149 subjects; at least about 150 subjects; at least about 151 subjects; at least about 152 subjects; at least about 153 subjects; at least about 154 subjects; at least about 155 subjects; at least about 156 subjects; at least about 157 subjects; at least about 158 subjects; at least about 159 subjects; at least about 160 subjects; at least about 161 subjects; at least about 162 subjects; at least about 163 subjects; at least about 164 subjects; at least about 165 subjects; at least about 166 subjects; at least about 167 subjects; at least about 168 subjects; at least about 169 subjects; at least about 170 subjects; at least about 171 subjects; at least about 172 subjects; at least about 173 subjects; at least about 174 subjects; at least about 175 subjects; at least about 176 subjects; at least about 177 subjects; at least about 178 subjects; at least about 179 subjects; at least about 180 subjects; at least about 181 subjects; at least about 182 subjects; at least about 183 subjects; at least about 184 subjects; at least about 185 subjects; at least about 186 subjects; at least about 187 subjects; at least about 188 subjects; at least about 189 subjects; at least about 190 subjects; at least about 191 subjects; at least about 192 subjects; at least about 193 subjects; at least about 194 subjects; at least about 195 subjects; at least about 196 subjects; at least about 197 subjects; at least about 198 subjects; at least about 199 subjects; or at least about 200 subjects. Recitation of each of these discrete values is understood to include ranges between each value.

A control sample can provide a reference value. For example, the reference value of the C22 to C16 ratio of the control sample or human control sample can be between about 0 and 50. For example, the reference value for C22/C16 can be about 0.01; about 0.02; about 0.03; about 0.04; about 0.05; about 0.06; about 0.07; about 0.08; about 0.09; about 0.1; about 0.2; about 0.3; about 0.4; about 0.5; about 0.6; about 0.7; about 0.8; about 0.9; about 1; about 1.5; about 2; about 2.5; about 3; about 3.5; about 4; about 4.5; about 5; about 5.5; about 6; about 6.5; about 7; about 7.5; about 8; about 8.5; about 9; about 9.5; about 10; about 10.5; about 11; about 11.5; about 12; about 12.5; about 13; about 13.5; about 14; about 14.5; about 15; about 15.5; about 16; about 16.5; about 17; about 17.5; about 18; about 18.5; about 19; about 19.5; about 20; about 20.5; about 21; about 21.5; about 22; about 22.5; about 23; about 23.5; about 24; about 24.5; about 25; about 25.5; about 26; about 26.5; about 27; about 27.5; about 28; about 28.5; about 29; about 29.5; about 30; about 30.5; about 31; about 31.5; about 32; about 32.5; about 33; about 33.5; about 34; about 34.5; about 35; about 35.5; about 36; about 36.5; about 37; about 37.5; about 38; about 38.5; about 39; about 39.5; about 40; about 40.5; about 41; about 41.5; about 42; about 42.5; about 43; about 43.5; about 44; about 44.5; about 45; about 45.5; about 46; about 46.5; about 47; about 47.5; about 48; about 48.5; about 49; about 49.5; or about 50. Recitation of each of these discrete values is understood to include ranges between each value. Recitation of each of a range is understood to include discrete values within the range.

The reference value of the ratio of C24/C16 of the control sample or human control sample can be between about 0 and 50. For example, the reference value for C24/C16 can be about 0.01; about 0.02; about 0.03; about 0.04; about 0.05; about 0.06; about 0.07; about 0.08; about 0.09; about 0.1; about 0.2; about 0.3; about 0.4; about 0.5; about 0.6; about 0.7; about 0.8; about 0.9; about 1; about 1.5; about 2; about 2.5; about 3; about 3.5; about 4; about 4.5; about 5; about 5.5; about 6; about 6.5; about 7; about 7.5; about 8; about 8.5; about 9; about 9.5; about 10; about 10.5; about 11; about 11.5; about 12; about 12.5; about 13; about 13.5; about 14; about 14.5; about 15; about 15.5; about 16; about 16.5; about 17; about 17.5; about 18; about 18.5; about 19; about 19.5; about 20; about 20.5; about 21; about 21.5; about 22; about 22.5; about 23; about 23.5; about 24; about 24.5; about 25; about 25.5; about 26; about 26.5; about 27; about 27.5; about 28; about 28.5; about 29; about 29.5; about 30; about 30.5; about 31; about 31.5; about 32; about 32.5; about 33; about 33.5; about 34; about 34.5; about 35; about 35.5; about 36; about 36.5; about 37; about 37.5; about 38; about 38.5; about 39; about 39.5; about 40; about 40.5; about 41; about 41.5; about 42; about 42.5; about 43; about 43.5; about 44; about 44.5; about 45; about 45.5; about 46; about 46.5; about 47; about 47.5; about 48; about 48.5; about 49; about 49.5; or about 50. Recitation of each of these discrete values is understood to include ranges between each value. Recitation of each of a range is understood to include discrete values within the range.

As described herein, the ceramide measures increased the C-statistic when added to standard coronary risk factors, indicating that they are new, independent risk factors. The C-statistic (sometimes called the “concordance” statistic or C-index) is a measure of goodness of fit for binary outcomes in a logistic regression model. In clinical studies, the C-statistic gives the probability a randomly selected patient who experienced an event (e.g., a disease or condition) had a higher risk score than a patient who had not experienced the event. It is equal to the area under the Receiver Operating Characteristic (ROC) curve and ranges from 0.5 to 1. Generally, a value below 0.5 can indicate a very poor model; a value of 0.5 can indicate that the model is no better than predicting an outcome than random chance; values over 0.7 can indicate a good model; values over 0.8 can indicate a strong model; and a value of 1 can indicate that the model perfectly predicts those group members who will experience a certain outcome and those who will not.

As described herein, the hazard ratio is calculated for coronary heart disease (CHD), heart failure (HF), and all-cause mortality.

In survival analysis, the hazard ratio (HR) is the ratio of the hazard rates corresponding to the conditions described by two levels of an explanatory variable. For example, in a drug study, the treated population may die at twice the rate per unit time as the control population. The hazard ratio would be 2, indicating higher hazard of death from the treatment. Or in another study, men receiving the same treatment may suffer a certain complication ten times more frequently per unit time than women, giving a hazard ratio of 10.

Hazard ratios can differ from relative risks and odds ratios in that RRs and ORs are cumulative over an entire study, using a defined endpoint, while HRs represent instantaneous risk over the study time period, or some subset thereof. Hazard ratios suffer somewhat less from selection bias with respect to the endpoints chosen and can indicate risks that happen before the endpoint.

Regression models can be used to obtain hazard ratios and their confidence intervals (see e.g., Spruance et al. 2004 Antimicrobial Agents and Chemotherapy 48 8 2787-2792).

As described herein, a subject suspected of having or a subject diagnosed with a cardiovascular-related disease, disorder, or condition can be treated by any method known in the art suitable for treating the disease. Therapeutic agents and methods of treating a cardiovascular-related disease, disorder, or condition are well known in the art.

For example, a therapeutic agent can be any agent suitable for treating a cardiovascular-related disease, disorder, or condition or any agent suitable to avoid cardiovascular mortality or non-cardiovascular mortality.

As another example, a therapeutic agent can be an ACE inhibitor (e.g., a vasodilator, which opens blood vessels more fully and can help reduce high blood and slow heart failure), an anti-arrhythmic medication (helps restore a normal pumping rhythm to the heart), antibiotics (help to prevent the onset of infections), anticoagulants (“blood thinners” to reduce the risk of developing blood clots from poorly circulating blood around faulty heart valves), beta-blockers (can reduce the heart's workload by helping the heart beat slower, reduce palpitations), calcium channel blockers, diuretics (“water pills” to reduces amount of fluid in the tissues and bloodstream which can lessen the workload on the heart), vasodilators (can lower the heart's work by opening and relaxing the blood vessels; reduced pressure may encourage blood to flow in a forward direction, rather than being forced backward through a leaky valve), a thrombolytic agent, an anticonvulsant agent, an anti-platelet agent, an anti-coagulant agent or a hematologic agent, an analgesic, a beta blocker or alpha activity agent, an ACE inhibitor, a calcium channel blocker, a vasodilator, a cholesterol-lowering and blood-pressure-lowering medicine, a blood pressure medicine, or medicines used to treat depression and pain.

As another example, a therapeutic agent can be an anticoagulant such as Warfarin (for example, Coumadin, Jantoven), Heparin, Dabigatran (Pradaxa), Rivaroxaban (Xarelto), Apixaban (Eliquis), or Edoxaban (Savaysa). As another example, a therapeutic agent can be a thrombolytic such as an IV tissue plasminogen activator (TPA) or Alteplase (Activase). As another example, a therapeutic agent can be an antiplatelet medication such as aspirin (for example, Bayer) or aspirin combined with dipyridamole (Aggrenox), Clopidogrel (Plavix), Prasugrel (Effient), or Ticagrelor (Brilinta). As another example, a therapeutic agent can be a cholesterol-lowering and blood-pressure-lowering medicines such as a statin (e.g., Atorvastatin (Lipitor), Rosuvastatin (Crestor)), angiotensin II receptor blockers (ARBs), angiotensin-converting enzyme (ACE) inhibitors, beta-blockers, calcium channel blockers, diuretics, Nicotinic Acids (e.g., Lovastatin (Advicor)), Cholesterol Absorption Inhibitors (e.g., Ezetimibe/Simvastatin (Vytorin)). As another example, a therapeutic agent can be a diuretic such as Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol, or Spironolactone (Aldactone). As another example, a therapeutic agent can be a medicine used to treat depression and pain such as amitriptyline, bupropion (Wellbutrin), citalopram (Celexa), fluoxetine (Prozac), sertraline (Zoloft), venlafaxine (Effexor). As another example, a therapeutic agent can be an anticonvulsant such as Diazepam (Valium) or Lorazepam (Ativan). As another example, a therapeutic agent can be an analgesic such as acetaminophen (Tylenol, Feverall, Aspirin Free Anacin). As another example, a therapeutic agent can be a beta blocker or alpha activity medication such as Labetalol (Normodyne, Trandate), Acebutolol (Sectral), Atenolol (Tenormin), Betaxolol (Kerlone), Bisoprolol/hydrochlorothiazide (Ziac), Bisoprolol (Zebeta), Metoprolol (Lopressor, Toprol XL), Nadolol (Corgard), Propranolol (Inderal), or Sotalol (Betapace). As another example, a therapeutic agent can be a digitalis preparation (e.g., Digoxin, Digitoxin) such as Lanoxin. As another example, a therapeutic agent can be a combined alpha and beta blocker such as carvedilol or labetalol hydrochloride. As another example, a therapeutic agent can be an ACE Inhibitor such as Enalapril (Vasotec), Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), or Trandolapril (Mavik). As another example, a therapeutic agent can be a calcium channel blocker such as Nicardipine (Cardene), Amlodipine (Norvasc, Lotrel), Diltiazem (Cardizem, Tiazac), Felodipine (Plendil), Nifedipine (Adalat, Procardia), Nimodipine (Nimotop), Nisoldipine (Sular), or Verapamil (Calan, Verelan). As another example, a therapeutic agent can be a vasodilator such as Nitroprusside sodium (Nipride, Nitropress, Sodium Nitroprusside), Isosorbide dinitrate (Isordil), Nesiritide (Natrecor), Hydralazine (Apresoline), Nitrates, or Minoxidil. As another example, a therapeutic agent can be an Angiotensin-2 Receptor Blockers (ARBs) or Angiotensin-2 Receptor Antagonists such as Candesartan (Atacand), Eprosartan (Teveten), Irbesartan (Avapro), Losartan (Cozaar), Telmisartan (Micardis), or Valsartan (Diovan). As another example, a therapeutic agent can be an Angiotensin-Receptor Neprilysin Inhibitor (ARNI) (a new drug combination of a neprilysin inhibitor and an ARB) such as Sacubitril/valsartan (Entresto).

As another example, a therapeutic agent can be an agent that modulates ceramide levels.

Provided herein is a process of treating a subject with a reduced C22:0 to C16:0 ratio or a reduced C24:0 to C16:0 ratio with an effective amount of a therapeutic agent or therapy, so as to treat a cardiovascular-related disease, disorder, or condition or avoid cardiac related death or all-cause death.

As described herein, a subject suspected of having or diagnosed with cardiovascular-related disease, disorder, or condition or is determined to be at risk for death can be treated by any therapeutic intervention or method known in the art. Therapeutic intervention can include any therapeutic agent or method of treating a cardiovascular-related disease, disorder, or condition known in the art.

For example, a therapeutic agent, as described herein, can be any agent to treat a cardiovascular-related disease, disorder, or condition or avoid cardiovascular mortality or non-cardiovascular mortality known in the art.

As described herein, a subject at increased risk for death or suspected of having or diagnosed with cardiovascular-related disease, disorder, or condition can be treated by any method known in the art. Methods of treating a cardiovascular-related disease, disorder, or condition are well known in the art. For example, treatments for a cardiovascular-related disease, disorder, or condition can include therapies such as procedures and surgeries well known in the art. For example, a procedure for the treatment of a cardiovascular-related disease, disorder, or condition can be angioplasty (e.g., laser), Artificial Heart Valve Surgery, Atherectomy, Bypass Surgery, Cardiomyoplasty, Heart Transplant, Minimally Invasive Heart Surgery, Radiofrequency Ablation, Stent Placement, or Transmyocardial Revascularization (TMR).

Methods described herein are generally performed on a subject in need thereof. A subject in need of the therapeutic methods described herein can be a subject having, diagnosed with, suspected of having, or at risk for developing heart failure or coronary heart disease or at increased risk of death. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a therapeutic agent is, for example, that amount that would cause the desired therapeutic effect in a subject while minimizing undesired side effects. In various embodiments, an effective amount of a therapeutic agent described herein can substantially inhibit heart failure, coronary heart disease, or cardiac related death, slow the progress of heart failure or coronary heart disease or limit the development of heart failure or coronary heart disease.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit heart failure, coronary heart disease, or cardiac related death, slow the progress of heart failure or coronary heart disease or limit the development of heart failure or coronary heart disease.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shawl (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a therapeutic agent can occur as a single event or over a time course of treatment. For example, a therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a cardiovascular disease, disorder, or condition.

A therapeutic agent can be administered simultaneously or sequentially with another agent, such as an antibiotic, an anti-inflammatory, or another agent. For example, a therapeutic agent can be administered simultaneously with another agent, such as an antibiotic or an anti-inflammatory. Simultaneous administration can occur through administration of separate compositions, each containing one or more of a therapeutic agent, an antibiotic, an anti-inflammatory, or another agent. Simultaneous administration can occur through administration of one composition containing two or more of a therapeutic agent, an antibiotic, an anti-inflammatory, or another agent. A therapeutic agent can be administered sequentially with an antibiotic, an anti-inflammatory, or another agent. For example, a therapeutic agent can be administered before or after administration of an antibiotic, an anti-inflammatory, or another agent.

### II. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a biological sample of a subject, ceramide containing calibration standards, a blank, a blank with internal standards, QC samples (LQC, MQC, and HQC), or BSA solutions. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Ceramide Quantification Assay

A liquid chromatography/mass spectrometry assay was developed to quantify plasma C24:0, C22:0, and C16:0 ceramides and the assay was used to determine ratios of very long chain to long chain ceramides in 2,642 Framingham Heart Study (FHS) participants and in 3,135 Study of Health in Pomerania (SHIP) participants.

Quantification of Ceramides

A fully validated liquid chromatography-tandem mass spectrometry assay was developed to quantify C24:0, C22:0, and C16:0 ceramides in frozen fasting plasma samples. A two-dimensional liquid chromatography-tandem mass spectrometry (LC-MS/MS) method for quantification of C24:0, C22:0, and C16:0 ceramides was developed according to FDA guidance for bioanalytical method validation.

Standard Curves and Quality Control (QC) Samples

Because of the endogenous presence of C16:0, C22:0, and C24:0 in human plasma, 5% bovine serum albumin (BSA) aqueous solution was used to prepare the calibration standards. Calibration curves were prepared by spiking the C16:0, C22:0, and C24:0 working solution into 5% BSA solution, and preparing serial dilutions that yielded eight calibration standards (0.01/0.04/0.1, 0.02/0.08/0.2, 0.05/0.2/0.5, 0.1/0.4/1, 0.2/0.8/2, 0.5/2/5, 1/4/10, and 2/8/20 μg/mL of C16:0/C22:0/C24:0 ceramides). 5% BSA solution served as blank. The standard curves prepared in 5% BSA solution were parallel to those prepared in human plasma, suggesting that the responsiveness of these ceramides in different matrices were the same and a calibration curve prepared in surrogate matrix was suitable for analysis of human plasma samples.

The pooled human plasma was analyzed to establish the mean concentration of endogenous C16:0, C22:0, and C24:0 ceramides. Low (LQC), middle (MQC), high (HQC), and dilution (DQC) quality control samples (endogenous level+0/0/0 μg/mL, endogenous level+0.75/3/7.5 μg/mL, endogenous level+1.5/6/15 μg/mL, and endogenous level+3/12/30 μg/mL) were prepared. The ceramides in the DQC samples were higher than the highest standard (2/8/20 μg/mL of C16:0/C22:0/C24:0 ceramides). The DQC sample was diluted 1:4 with 5% BSA solution, prior to extraction.

Sample Preparation

Standards, QCs, blank, or study samples (50 μL) were aliquoted into a 96-well (2 mL/well) plate. To each well, 400 μL of internal standards/protein precipitation solution (0.025/0.025/0.0625 μg/mL of d5-C16:0, d4-C22:0, and d4-C24:0 ceramides in isopropanol-chloroform (9:1) was added and 400 μL of isopropanol-chloroform (9:1) was used for a blank. The plate was vortexed for 3 min, centrifuged for 10 min at 3000 g, and 250 μL of supernatant transferred to clean 96 wells (1 mL/well) plate with a Tomtec Quadra 96 (Tomtec, Hamden, Conn.) for LC-MS/MS assay.

LC-MS/MS Analysis

LC-MS/MS analysis was conducted on a Shimadzu (Columbia, Md.) Prominence HPLC system coupled with an Applied Biosystems/MDS Sciex (Ontario, Canada) 4000QTRAP mass spectrometer using multiple reaction monitoring (MRM). The HPLC system consists of Prominence HPLC system with a CBM-20A system controller, 4 LC-20AD pumps, a SIL-20ACHT autosampler, a DGU-20A5R degasser, and a rack changer.

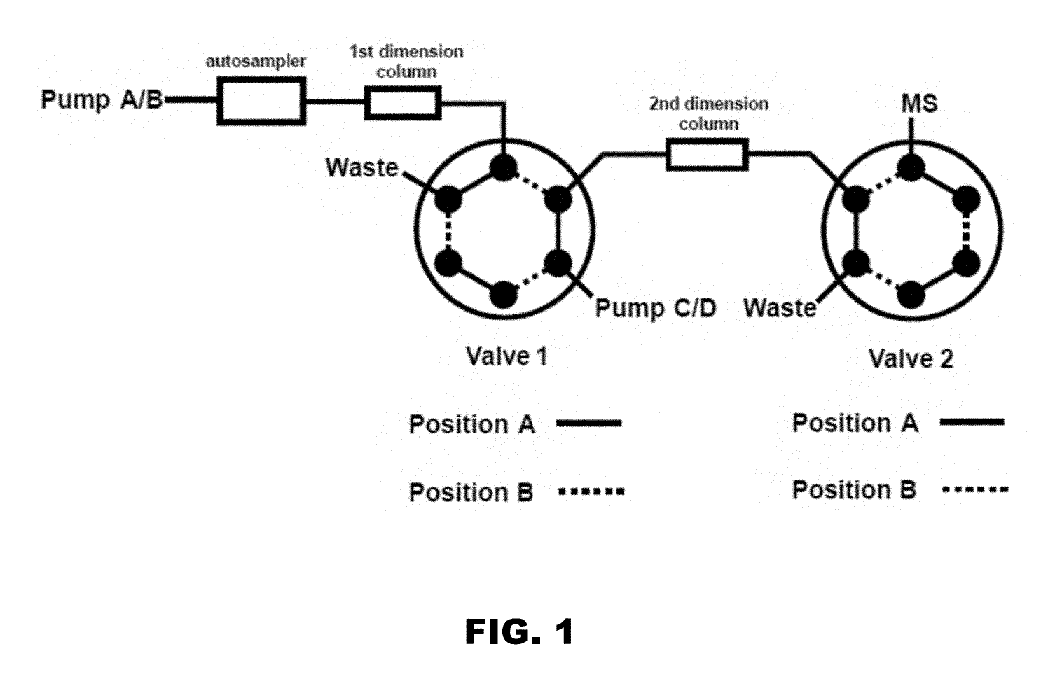

The chromatography was performed using an Atlantis HILIC silica column (3×50 mm, 3 μm; Waters, Milford, Mass.) as the first dimension at ambient temperature and Xselect HSS C18 (4.6×50 mm, 3.5 μm; Waters, Milford, Mass.) as the second dimension at ambient temperature. The compartments of the autosampler and rack changer were set at 4° C. FIG. 1 is a schematic of the column and switching valve arrangement for 2D-LC. For the first dimension LC, mobile phase A (0.1% formic acid in water) and mobile phase B (0.1% formic acid in acetonitrile) were operated with a gradient elution as follows: 0-1.0 min 95% B, 1.0-1.2 min 95-50% B, 1.2-2.4 min 50% B, 2.4-2.5 min 50-95% B, and 2.5-5.0 min 95% B at a flow rate of 0.6 mL/min. The solvent gradient for second dimension LC using 0.1% formic acid in water (phase C) and 0.1% formic acid in isopropanol-acetonitrile (1:2) (phase D) at a flow rate of 1 mL/min was as follows: 0-0.9 min 95% D, 0.9-3.0 min 95-100% D, 3.0-4.5 min 100% D, 4.5-4.6 min 100-95% D, and 4.6-5.0 min 95% D. Valve 1 was kept at the A position during 0-0.5 min and 0.9-5.0 min, and at the B position during 0.5-0.9 min. Valve 2 was kept at the A position during 0-2.0 min and 3.7-5.0 min, and at the B position during 2.0-3.7 min. The injection volume was 5 μL. The ESI source temperature was 400° C. The ESI spray voltage was 5500 V. For all the ceramides and their internal standards, the declustering potential, entrance potential, and the collision cell exit potential were 66 V, 10 V, and 10 V, respectively. The collision and curtain gas were set at medium and 15, respectively. Both desolvation gas and nebulizing gas were set at 45 L/min. The collision energies for all the MRM transitions including m/z 538.5 to 264.3 (quantifier for C16:0), m/z 538.5 to 282.3 (qualifier for C16:0), m/z 622.6 to 264.3 (quantifier for C22:0), m/z 622.6 to 282.3 (qualifier for C22:0), m/z 650.6 to 264.3 (quantifier for C24:0), m/z 650.6 to 282.3 (qualifier for C24:0), m/z 543.5 to 264.3 (d5-C16:0), m/z 626.6 to 264.3 (d4-C22:0) and m/z 654.6 to 264.3 (d4-C24:0) were set at 40 eV. The dwell time was set at 50 ms for each mass transition. Data were acquired and analyzed by Analyst software (version 1.5.2). Calibration curves were constructed by plotting the corresponding peak area ratios of analyte/internal standard versus the corresponding analyte concentrations using weighted (1/x2) least-squares regression analysis.

Analysis of Clinical Samples

Samples analyzed consisted of calibration standards in duplicate, a blank, a blank with internal standards, QC samples (LQC, MQC, and HQC), and unknown clinical samples. The total number of QC samples was at least 5% of that of unknown clinical samples. The standard curve covered the expected unknown sample concentration range, and samples that exceeded the highest standard could be diluted and re-assayed. In the dilution sample re-assay, a diluted QC in triplicate is also included in the analytical run. The LC-MS/MS run acceptance criteria included: 1) a minimum of six standards within ±15%, except for the lowest standard for which ±20% of the nominal value was accepted; 2) at least 67% of the QC samples within 15% of their respective nominal values; and 3) not all replicates at the same level of QC outside ±15% of the nominal value. The analysis for FHS and SHIP samples was performed in 16 and 7 batches, respectively. All batches met acceptance criteria.

Healthy Volunteers

Posted advertisements were used to recruit healthy, non-smoking men (n=12) and women (n=12), ages 40 to 60 years (mean age 41), at Washington University School of Medicine. Subjects were free of hypertension, diabetes (hemoglobin A1c<6.5% and normal glucose tolerance test), cardiovascular disease (normal stress echocardiogram), and other major systemic illness. Morning fasting plasma was obtained in venous blood draws two weeks apart. The range and mean values at each time point were determined. Percent change was calculated per subject and Student's one sample t-test was used to determine if mean percent change differed from zero.

FHS Samples

Participants were evaluated from the Framingham Heart Study (FHS) Offspring cohort who attended the eighth examination cycle (2005-2008) when plasma samples were obtained. The Boston University Medical Center and Washington University Institutional Review Boards approved this study protocol, and all subjects provided written informed consent.

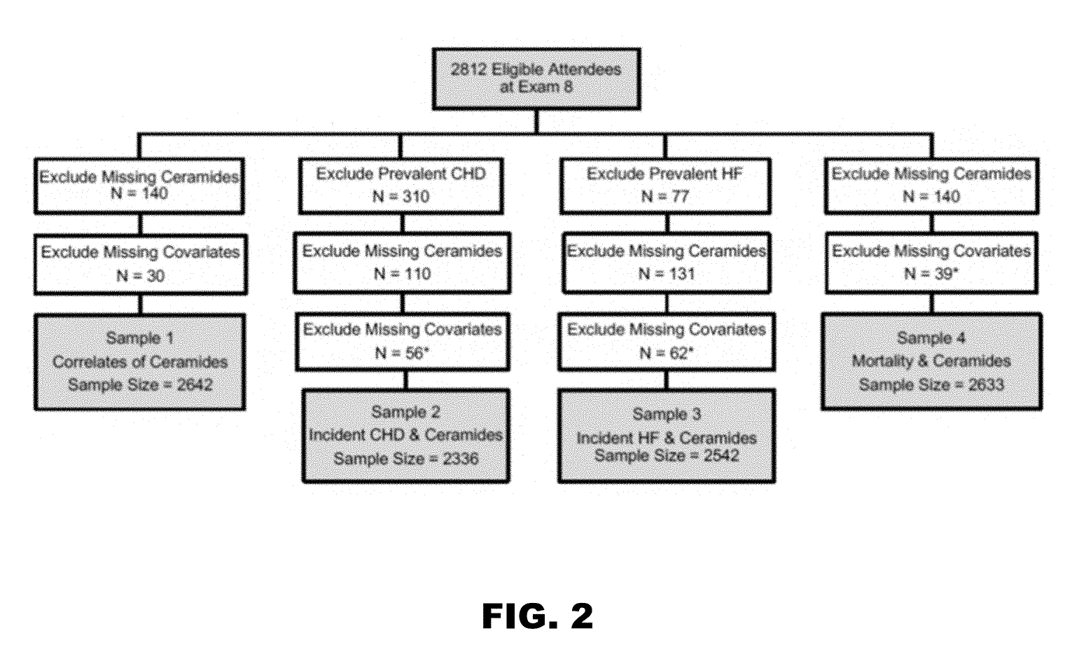

From a total of 2,812 participants at the 8^{th} examination cycle, four different participant samples were used for analyses (see e.g., FIG. 2). Sample 1 was used to examine clinical correlates of ceramides and excluded individuals who were missing plasma samples (n=140) or missing covariates (n=30), giving a final sample size of 2,642. From this, additional samples to examine outcomes of interest excluded those with the prevalent disease of interest or missing follow-up time. Sample 2 (n=2,336) examined incident CHD, which includes myocardial infarction, coronary insufficiency, and angina pectoris. Sample 3 (n=2,542) examined incident HF. Sample 4 (n=2,633) examined mortality. Criteria for these events, adjudication process, and criteria for co-variates have been previously published (see Kannel et al. 1987. Health and Human Services, Bethesda, Md., Publication NIH 87-2703) and all events were adjudicated during the follow-up period from baseline through 2012.

SHIP Samples

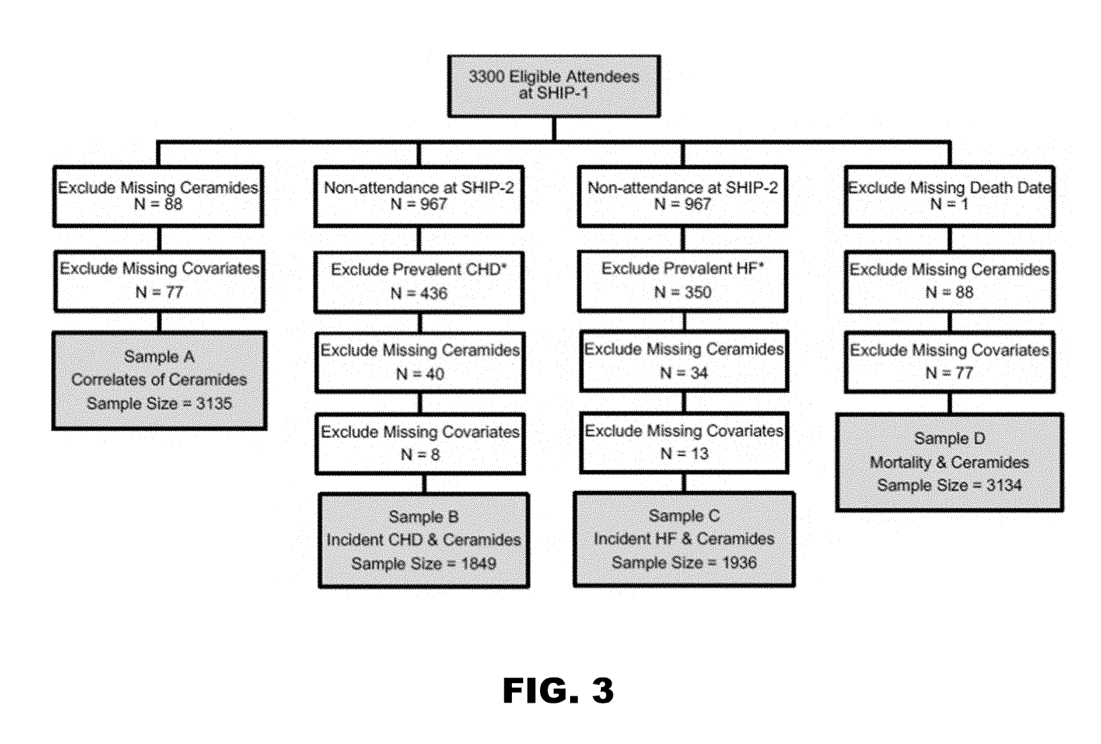

Data and plasma samples were used from the first cohort of the Study of Health in Pomerania (SHIP), a northern European community-based study (see Dorr et al. 2005 J Clin Endocrinol Metab. 90 673-7). The study was approved by the Ethics Committee of the University Medicine Greifswald, and all subjects provided written informed consent. CHD and HF events were evaluated between the second and third examination cycle, SHIP-1 (2002-2006) and SHIP-2 (2008-2012), while mortality was tracked from SHIP-1 through the most recent mortality survey in March 2016. From the 3,300 participants who attended SHIP-1, who had plasma samples obtained, 88 were excluded for missing ceramide values and 77 were removed for missing covariates, yielding Sample A (n=3,135, see e.g., FIG. 3) that was used to assess clinical correlates. Samples B (examined CHD, n=1,849) and C (examined HF, n=1,936) were created from the 2,333 participants who also attended SHIP-2, when CHD and HF status was reassessed. Sample D, used to assess mortality, is the same as Sample A but with one additional individual removed due to a missing death date (n=3,134). These samples mirror samples 2, 3, and 4 in the FHS analysis (with exclusions for prevalent disease of interest, unknown disease status at SHIP-1 or SHIP-2 examinations, unknown follow-up time, missing ceramide values, or missing covariates). In these analyses, definitions for events and criteria for co-variates were aligned to those used in FHS.

Analysis of FHS and SHIP Samples

Utilizing data from FHS sample 1 participants, multiple linear regression models were fit to assess the correlates of the C24:0/C16:0 and C22:0/C16:0 ceramide ratios and C24:0, C22:0, and C16:0 ceramide levels (separate models for each ratio or lipid). In these models, ceramide levels served as the dependent variable, and age, sex, BMI, systolic blood pressure (SBP), antihypertensive medications, smoking status, diabetes, the ratio of total to HDL cholesterol, and lipid-lowering medication served as independent variables. Models for all-cause mortality were additionally adjusted for prevalent CVD. After confirming that the proportional hazards assumption was satisfied, data from participants in samples 2, 3, and 4 were used to perform Cox regression, evaluating the association of ceramide ratios or ceramide levels with each of: CHD, HF, all-cause mortality, CVD mortality, and non-CVD mortality (separate models for each event and for each ceramide ratio or species). For all multivariable models, adjustments were made for age, sex, BMI, SBP, diabetes, smoking status, antihypertensive medications, the ratio of total to HDL cholesterol, and lipid-lowering medication. All-cause mortality models were additionally adjusted for prevalent CVD. Furthermore, cumulative incidence plots were created to assess the incidence of events by ceramide ratio tertile. The incremental effect of ceramide ratios over standard CVD risk factors was assessed in FHS sample 4 by examining the change in c-statistics between models without vs. with ceramide ratios.

Analyses were repeated using SHIP samples A, B, C, and D. Cox proportional hazards regression models were used to examine the association between ceramides and mortality, since exact death dates were known. For non-fatal events, the midpoint between an individual's SHIP-1 and SHIP-2 dates was used as the follow-up time. When modeling the association between ceramides and CHD or HF, constant hazard models were utilized with a Poisson distribution and an offset equal to the log follow-up time, because exact times for non-fatal events is not available in SHIP. All models were adjusted for the same covariates used in FHS analyses.

Meta-analyses were performed using FHS and SHIP samples. Values of I² were calculated for each association to determine the degree of heterogeneity among the results. Maximum Likelihood Random Effect models were used in the meta-analyses to account for the moderate heterogeneity indicated by the values of I². Statistical significance was assessed using a P-value of <0.05. All FHS and meta-analyses were performed using SAS software version 9.3. (Cary, N.C.), while all SHIP analyses were performed using Stata version 14.2 (Stata Corp. 2015).

Results

Over a mean follow-up of 6 years in FHS, there were 88 coronary heart disease events and 239 deaths. Over a median follow-up time in SHIP of 5.75 years for coronary heart disease and 8.24 years for mortality, there were 209 coronary heart disease events and 377 deaths. In a meta-analysis of the two cohorts, C24:0/C16:0 ceramide ratios were inversely associated with coronary heart disease (hazard ratio per standard deviation increment [HR] 0.80, 95% CI [0.71, 0.91], P=0.0006). Moreover, the C24:0/C16:0 ceramide ratio was inversely associated with all-cause mortality (HR 0.64, 95% CI [0.58, 0.70], P<0.0001).

High Throughput Assay for Quantification of Ceramides.

A liquid chromatography-tandem mass spectrometry (LC-MS/MS) assay was developed for ceramides to simultaneously quantify C16:0, C22:0, and C24:0 ceramides. The linear dynamic ranges for C16:0, C22:0, and C24:0 ceramides in this triplex assay were 0.01-2, 0.04-8, and 0.1-20 μg/mL, respectively, which encompass the values reported for each of these ceramide species in human plasma. The intra- and inter-assay precisions were within 7.8%, 7.6%, and 6.9% coefficient of variation (CV) for C16:0, C22:0, and C24:0 ceramides, respectively. The intra- and inter-assay accuracy were within ±3.2%, ±4.5%, and ±4.9% deviation of the nominal concentration values for C16:0, C22:0 and C24:0 ceramides, respectively. The stability of C16:0, C22:0, and C24:0 ceramides were determined to be acceptable in human plasma following 5 freeze-thaw cycles (difference <5% for each). These data indicate that the triplex assay is accurate, precise, and rugged. An LC run time of 5 min/sample indicates this assay is suitable for high throughput applications.

The triplex assay was used to quantify C16:0, C22:0 and C24:0 ceramides and to calculate the ratios of C24:0/C16:0 and C22:0/C16:0 ceramides in fasting plasma samples obtained two weeks apart from 24 healthy, non-smoking volunteers who were free of diabetes, hypertension, and obstructive coronary heart disease. The range of C16:0, C22:0 and C24:0 ceramide values was within the linear range for each species (see e.g., TABLE 1).

TABLE 1
C16:0, C22:0 and C24:0 Ceramides and C24:0/C16:0 and C22:0/C16:0 Ceramide
Ratios in Healthy Volunteers

| | Time 1 | Time 1 | Time 2 | Time 2 | Mean % | | |
|---|---|---|---|---|---|---|---|
| Measure | range | mean | range | mean | change* | 95% CI | PValue† |
| C16:0 | 0.105-0.270 | 0.151 | 0.0612-0.187 | 0.129 | −12.6 | (−20.1, −5.0) | 0.002 |
| ceramide | | | | | | | |
| C22:0 | 0.291-0.979 | 0.547 | 0.156-0.988 | 0.502 | −8.8 | (−16.6, −1.0) | 0.028 |
| ceramide | | | | | | | |
| C24:0 | 0.879-3.50 | 2.04 | 0.735-3.08 | 1.82 | −9.8 | (−17.6, −2.0) | 0.017 |
| ceramide | | | | | | | |
| C24:0/C16:0 | 6.43-25.0 | 13.6 | 8.88-22.3 | 14.0 | 6.4 | (−4.2, 17.0) | 0.23 |
| ceramide | | | | | | | |
| ratio | | | | | | | |
| C22:0/C16:0 | 1.63-4.99 | 3.59 | 2.05-7.16 | 3.80 | 6.6 | (−1.9, 15.2) | 0.12 |
| ceramide | | | | | | | |
| ratio | | | | | | | |

Ceramides were quantified in fasting plasma obtained in blood draws at times 1 and 2 (2 weeks apart) from 24 human volunteers who were free of diabetes, hypertension, obstructive coronary heart disease and smoking. Ceramide values are reported in μg/ml; ceramide ratios have no units.
*The difference and percent change were calculated per subject and then aggregated and summarized by the mean difference and mean percent change, respectively. [Percent change = 100 * (Time 2 − Time 1)/Time 1 (calculated per patient)]
†Test to determine if percent change differs from 0%.

The C24:0/C16:0 ceramide ratio ranged from 6.43 to 25.0 and the C22:0/C16:0 ceramide ratio ranged from 1.63 to 7.16. The difference and percent change were calculated per subject and then aggregated and summarized by the mean difference and mean percent change, respectively. Between the samples drawn two weeks apart, the mean change in measures of C16:0, C22:0, and C24:0 ceramides was 12.6, 8.8 and 9.8%, respectively (P<0.05). By contrast, the mean percent changes in C24:0/C16:0 and C22:0/C16:0 ceramide ratios were only 6.4 and 6.6%, respectively, and were not significantly different. Thus, during an interval in which meaningful biological change was not expected in this small sample of healthy individuals, basal level of variation in the abundance of individual ceramide species is <15%, and very long chain to long chain ceramide ratios are relatively stable.

Associations Between Ceramide Ratios and Standard Risk Factors in FHS and SHIP.

Next C16:0, C22:0, and C24:0 ceramides were quantified in plasma from the Offspring cohort participants of FHS and from SHIP. Overall, FHS and SHIP participants were middle-aged to older individuals, and more than half the participants were women (see e.g., TABLE 2).

TABLE 2
Descriptive Characteristics of largest study samples
in FHS (Sample 1) and SHIP (Sample A) at baseline.

| | FHS | | SHIP | |
|---|---|---|---|---|
| Characteristics | (n = 2642) | | (n = 3135) | |
| Age, years | 66.2 ± 9.0 | | 54.0 ± 15.1 | |
| Men (%) | 1208 | (45.7) | 1508 | (48.1) |
| Body Mass Index, kg/m² | 28.3 ± 5.4 | | 27.9 ± 4.9 | |
| Systolic Blood Pressure, mm Hg | 128.4 ± 17.2 | | 132.2 ± 19.4 | |
| Diastolic Blood Pressure, mm Hg | 73.4 ± 10.1 | | 81.4 ± 10.5 | |
| Total Cholesterol, mg/dL | 186.1 ± 37.2 | | 214.4 ± 45.1 | |
| HDL Cholesterol, mg/dL | 57.4 ± 18.2 | | 45.6 ± 16.3 | |
| Plasma C16:0 Ceramide, μg/mL | 0.2 ± 0.04 | | 0.2 ± 0.05 | |
| Plasma C22:0 Ceramide, μg/mL | 0.6 ± 0.2 | | 0.7 ± 0.2 | |
| Plasma C24:0 Ceramide, μg/mL | 2.3 ± 0.6 | | 2.5 ± 0.7 | |
| Plasma C22:0/C16:0 Ceramide | 3.8 ± 0.8 | | 3.1 ± 0.6 | |
| Plasma C24:0/C16:0 Ceramide | 14.0 ± 3.4 | | 11.8 ± 2.6 | |
| Hypertension (%) | 1540 | (58.3) | 1983 | (63.3) |
| Antihypertensive Medication Use (%) | 1280 | (48.5) | 1292 | (41.2) |
| Lipid Lowering Medication Use (%) | 1128 | (42.7) | 457 | (14.6) |
| Diabetes mellitus (%) | 364 | (13.8) | 455 | (14.5) |
| Smokers (%) | 236 | (8.9) | 820 | (26.2) |

FHS = Framingham Heart Study
SHIP = Study of Health in Pomerania
Values are mean ± SD for continuous variables and n (%) for categorical variables.

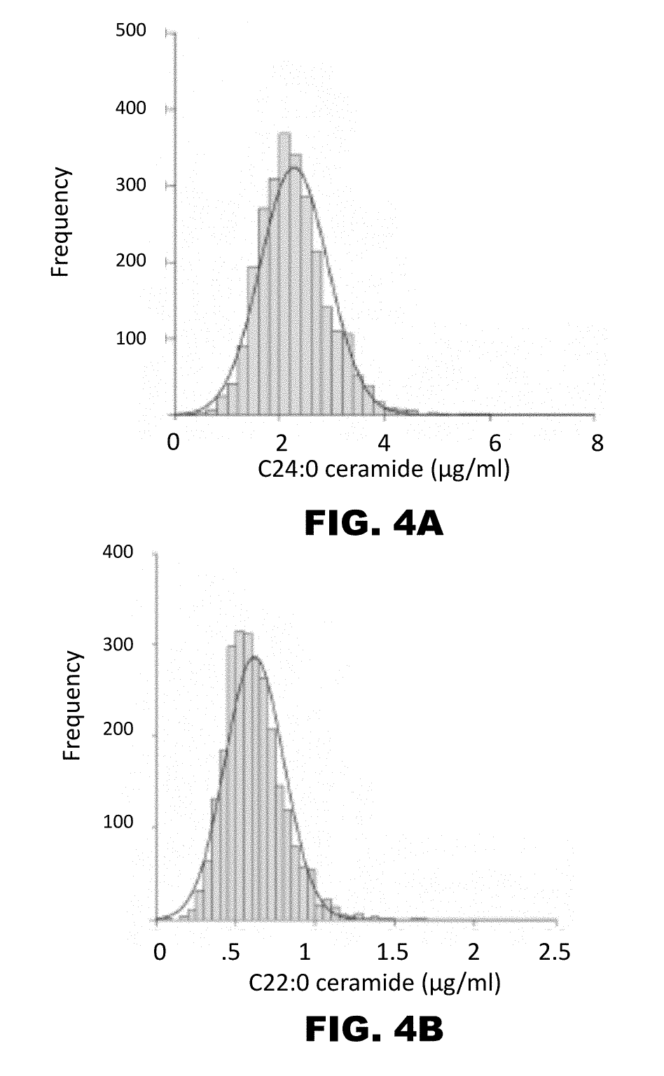

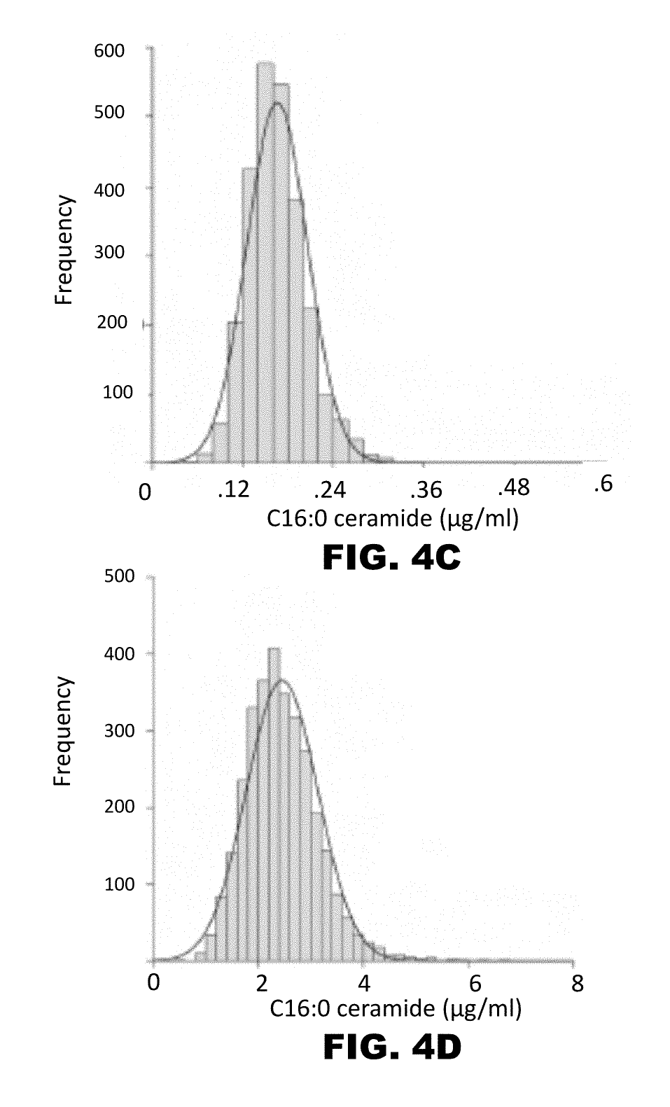

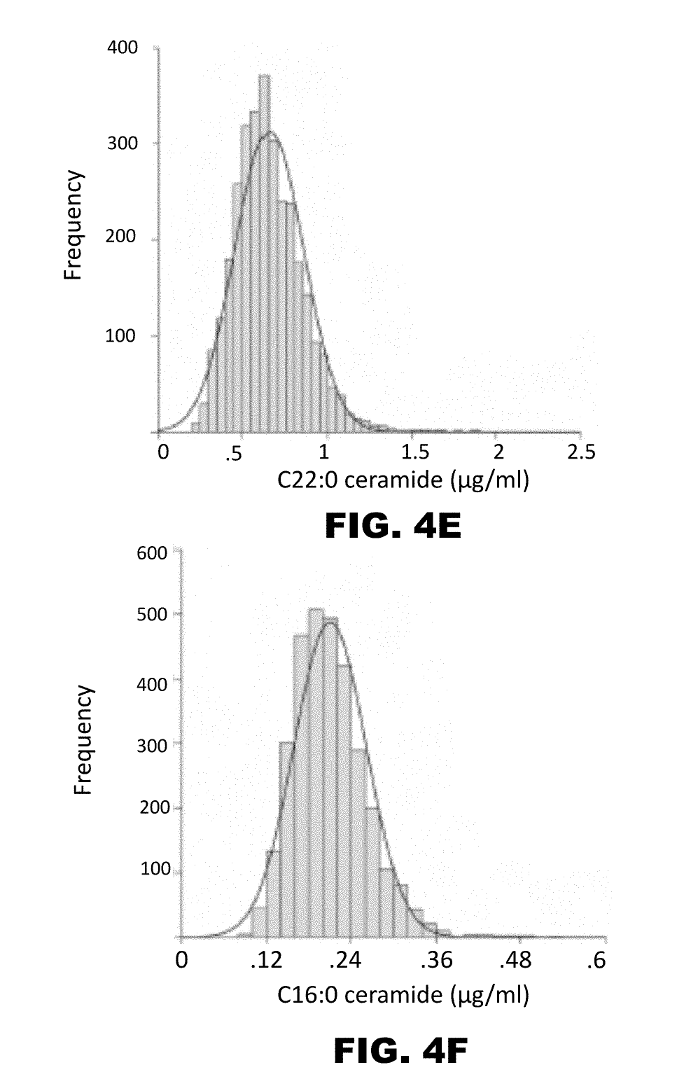

Values for ceramide species were normally distributed in both FHS and SHIP (see e.g., FIG. 4). C24:0 ceramide was nearly 4-fold more abundant than C22:0 ceramide, and 12-fold more abundant than C16:0 ceramide. These values were used to calculate the ratios of C24:0/C16:0 and C22:0/C16:0 ceramides.

In multiple linear regression models in both FHS and SHIP, age, use of antihypertensive medication, smoking status, and prior CVD were inversely associated with plasma C24:0/C16:0 ceramide ratio, whereas male sex and systolic blood pressure were directly associated with the ratio (all P<0.03, see e.g., TABLE 3).

TABLE 3
Clinical Correlates of Plasma C24:0/C16:0
Ceramide Ratios in FHS and SHIP

| | FHS | | SHIP | |
|---|---|---|---|---|
| Variable | βEstimate | PValue | βEstimate | PValue |
| Age | −0.085 | <0.0001 | −0.032 | <0.001 |
| Male | 0.670 | <0.0001 | 0.549 | <0.001 |
| Body Mass Index | −0.007 | 0.59 | −0.013 | 0.20 |
| Systolic Blood Pressure | 0.013 | 0.0008 | 0.014 | <0.001 |
| Antihypertensive Medication | −0.416 | 0.0039 | −0.269 | 0.021 |
| Smoking Status | −0.512 | 0.0232 | −0.238 | 0.026 |
| Diabetes Status | 0.333 | 0.09 | −0.007 | 0.96 |
| Total/HDL Cholesterol | 0.091 | 0.15 | 0.084 | <0.001 |
| Lipid Lowering Medication | 0.227 | 0.11 | 0.345 | 0.015 |
| Prevalent CVD | −0.649 | 0.0005 | −0.309 | 0.006 |

Multiple linear regression models were used, where the ceramide ratio served as the dependent variable and clinical correlates served as independent variables.
βEstimates represent the increase in plasma ceramide levels per-unit increase in continuous variables and for the presence (vs. absence) of dichotomous variables.
FHS = Framingham Heart Study
SHIP = Study of Health in Pomerania

The C24:0/C16:0 ratio was not related to diabetes status in either FHS or SHIP. In FHS and in SHIP, the C22:0/C16:0 ceramide ratio was inversely associated with age and directly associated with body mass index, systolic blood pressure, and total/HDL cholesterol, but was not associated with male sex (see e.g., TABLE 4).

TABLE 4
Clinical Correlates of Plasma C22:0/C16:0
Ceramide Ratios in FHS and SHIP

| | FHS | | SHIP | |
|---|---|---|---|---|
| Variable | βEstimate | PValue | βEstimate | PValue |
| Age | −0.012 | <0.0001 | −0.006 | <0.001 |
| Male | 0.008 | 0.81 | −0.028 | 0.24 |
| Body Mass Index | 0.015 | <0.0001 | 0.019 | <0.001 |
| Systolic Blood Pressure | 0.002 | 0.0242 | 0.002 | 0.002 |
| Antihypertensive Medication | −0.099 | 0.0048 | −0.021 | 0.45 |
| Smoking Status | −0.112 | 0.0428 | −0.024 | 0.35 |
| Diabetes Status | 0.217 | <0.0001 | 0.060 | 0.07 |
| Total/HDL Cholesterol | 0.172 | <0.0001 | 0.065 | <0.001 |
| Lipid Lowering Medication | 0.064 | 0.07 | 0.065 | 0.06 |
| Prevalent CVD | −0.119 | 0.0094 | −0.051 | 0.06 |

Multiple linear regression models were used, where the ceramide ratio served as the dependent variable and clinical correlates served as independent variables.
βEstimates represent the increase in ceramide levels for a unit increase in continuous variables and for presence vs. absence of dichotomous variables.
FHS = Framingham Heart Study,
SHIP = Study of Health in Pomerania

In FHS only, antihypertensive medication, smoking status, and prevalent CVD were inversely associated with C22:0/C16:0. Although the C22:0/C16:0 ratio was directly associated with diabetes status in FHS, this finding was not replicated in SHIP. Each ceramide species individually was inversely associated with male sex and use of antihypertensive medication (see e.g., TABLE 5).

TABLE 5
Clinical Correlates of Individual Plasma
Ceramide Species in FHS and SHIP

| | C16:0 Ceramide | | | |
|---|---|---|---|---|
| | FHS | | SHIP | |
| Variable | βEstimate | PValue | βEstimate | PValue |
| Age | 0.001 | <0.0001 | 0.001 | <0.001 |
| Male | −0.018 | <0.0001 | −0.018 | <0.001 |
| Body Mass Index | −0.001 | <0.0001 | −0.0008 | <0.001 |
| Systolic Blood Pressure | 0.00007 | 0.09 | 0.0002 | 0.001 |
| Antihypertensive Medication | −0.003 | 0.0429 | −0.008 | <0.001 |
| Smoking Status | 0.008 | 0.0007 | 0.009 | <0.001 |
| Diabetes Status | −0.002 | 0.27 | −0.007 | 0.003 |
| Total/HDL Cholesterol | 0.014 | <0.0001 | 0.010 | <0.001 |
| Lipid Lowering Medication | −0.018 | <0.0001 | −0.012 | <0.001 |
| Prevalent CVD | −0.001 | 0.60 | 0.00008 | 0.97 |
| | C22:0 Ceramide | | | |
| | FHS | | SHIP | |
| Variable | βEstimate | PValue | βEstimate | PValue |
| Age | 0.0002 | 0.67 | 0.002 | <0.001 |
| Male | −0.067 | <0.0001 | −0.065 | <0.001 |
| Body Mass Index | −0.001 | 0.0211 | 0.001 | 0.040 |
| Systolic Blood Pressure | 0.0007 | 0.0005 | 0.0009 | <0.001 |
| Antihypertensive Medication | −0.028 | <0.0001 | −0.026 | 0.001 |
| Smoking Status | 0.008 | 0.46 | 0.022 | 0.002 |
| Diabetes Status | 0.022 | 0.0187 | −0.008 | 0.40 |
| Total/HDL Cholesterol | 0.082 | <0.0001 | 0.047 | <0.001 |
| Lipid Lowering Medication | −0.056 | <0.0001 | −0.020 | 0.036 |
| Prevalent CVD | −0.021 | 0.0199 | −0.009 | 0.25 |
| | C24:0 Ceramide | | | |
| | FHS | | SHIP | |
| Variable | βEstimate | PValue | βEstimate | PValue |
| Age | −0.006 | <0.0001 | 0.005 | <0.001 |
| Male | −0.143 | <0.0001 | −0.107 | <0.001 |
| Body Mass Index | −0.015 | <0.0001 | −0.012 | <0.001 |
| Systolic Blood Pressure | 0.003 | <0.0001 | 0.005 | <0.001 |
| Antihypertensive Medication | −0.109 | <0.0001 | −0.135 | <0.001 |
| Smoking Status | 0.004 | 0.93 | 0.054 | 0.037 |
| Diabetes Status | 0.014 | 0.69 | −0.089 | 0.007 |
| Total/HDL Cholesterol | 0.207 | <0.0001 | 0.139 | <0.001 |
| Lipid Lowering Medication | −0.205 | <0.0001 | −0.061 | 0.07 |
| Prevalent CVD | −0.110 | 0.0010 | −0.052 | 0.058 |

Multiple linear regression models were used, where ceramides served as dependent variables and clinical correlates served as independent variables; beta estimates represent the increase in ceramide levels for a unit increase in continuous variables and for presence vs. absence of dichotomous variables.
FHS = Framingham Heart Study,
SHIP = Study of Health in Pomerania

Association Between Ceramide Ratios and Incidence of CHD, HF, and all-Cause Mortality.

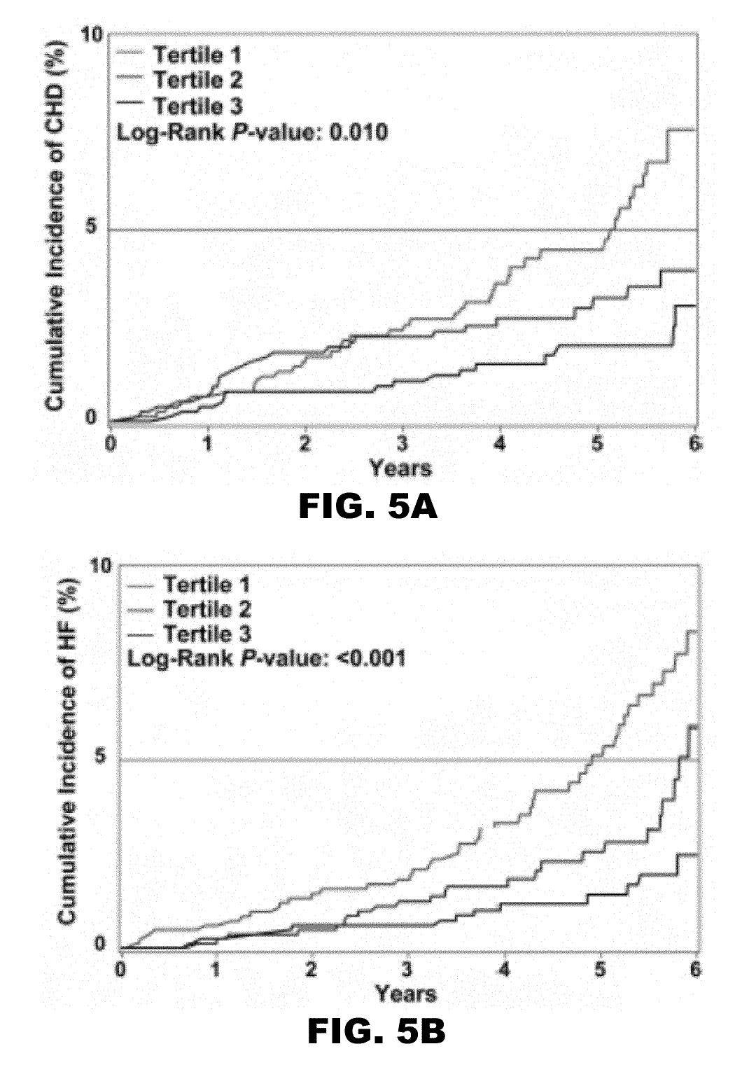

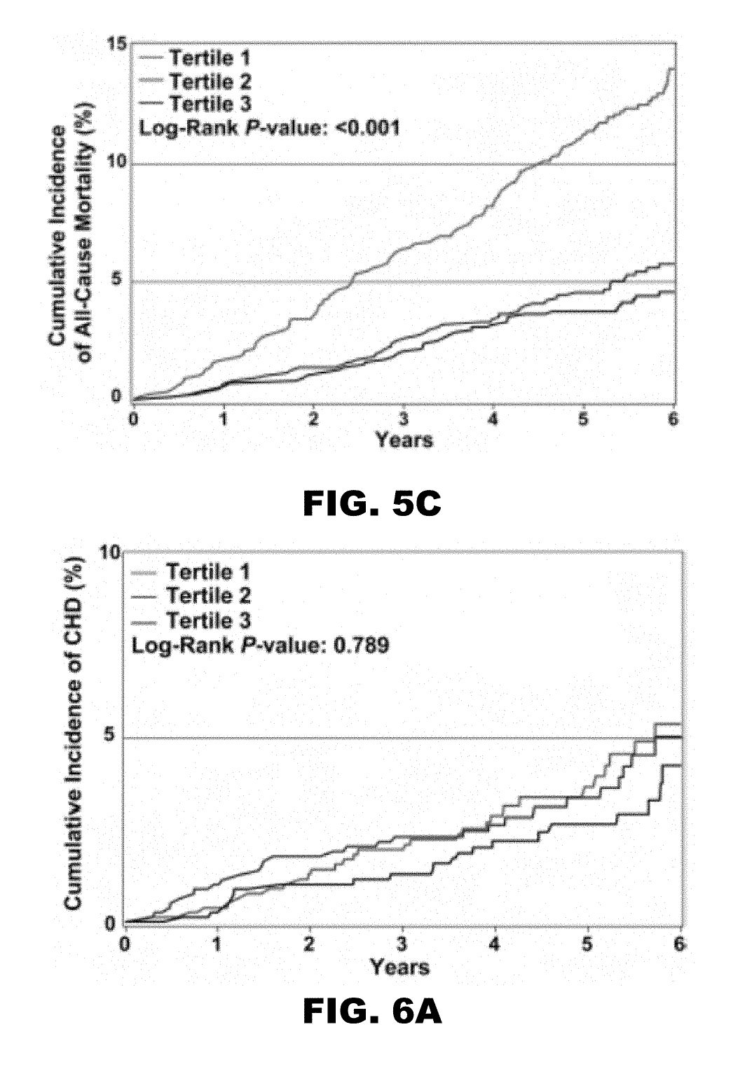

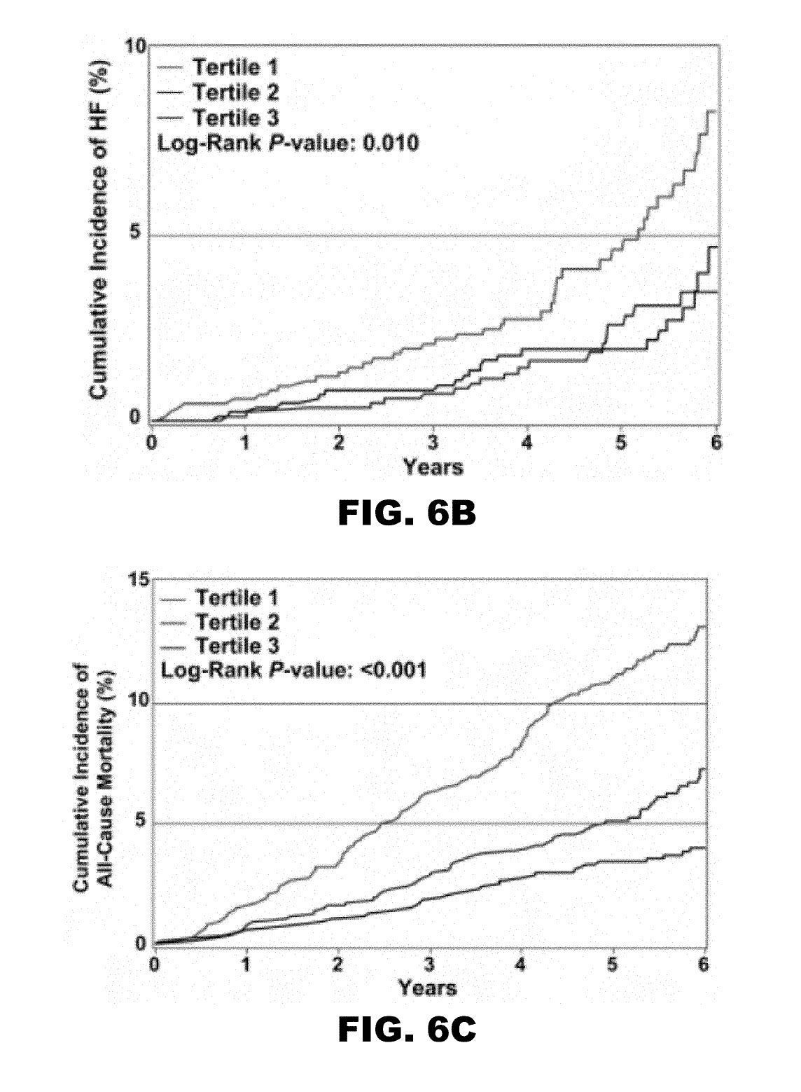

In FHS, there were 88 CHD and 90 HF events, as well as 239 deaths during a mean follow-up period of 6 years. In SHIP, there were 209 CHD and 146 HF events over a median follow-up time of 5.75 years, and 377 deaths over a median follow-up of 8.24 years. Cumulative incidence of CHD, HF, and all-cause mortality decreased across ceramide 24:0/16:0 tertiles in FHS, with the highest incidence in the lowest ceramide ratio tertile (see e.g., FIG. 5). The increase in median ceramide 24:0/16:0 ratio across tertiles was large (81% increase from tertile 1 to 2, 62% increase from tertile 2 to 3) compared to the differences observed in repeated measures of the ratio determined at two week intervals (6%). Similarly, cumulative incidence of HF and all-cause mortality, but not CHD, decreased across ceramide 22:0/16:0 tertiles (see e.g. FIG. 6). Together, these findings indicate that in FHS, individuals with the lowest ratios of very long chain to long chain ceramides were at greatest risk for CHD, HF, and all-cause mortality.

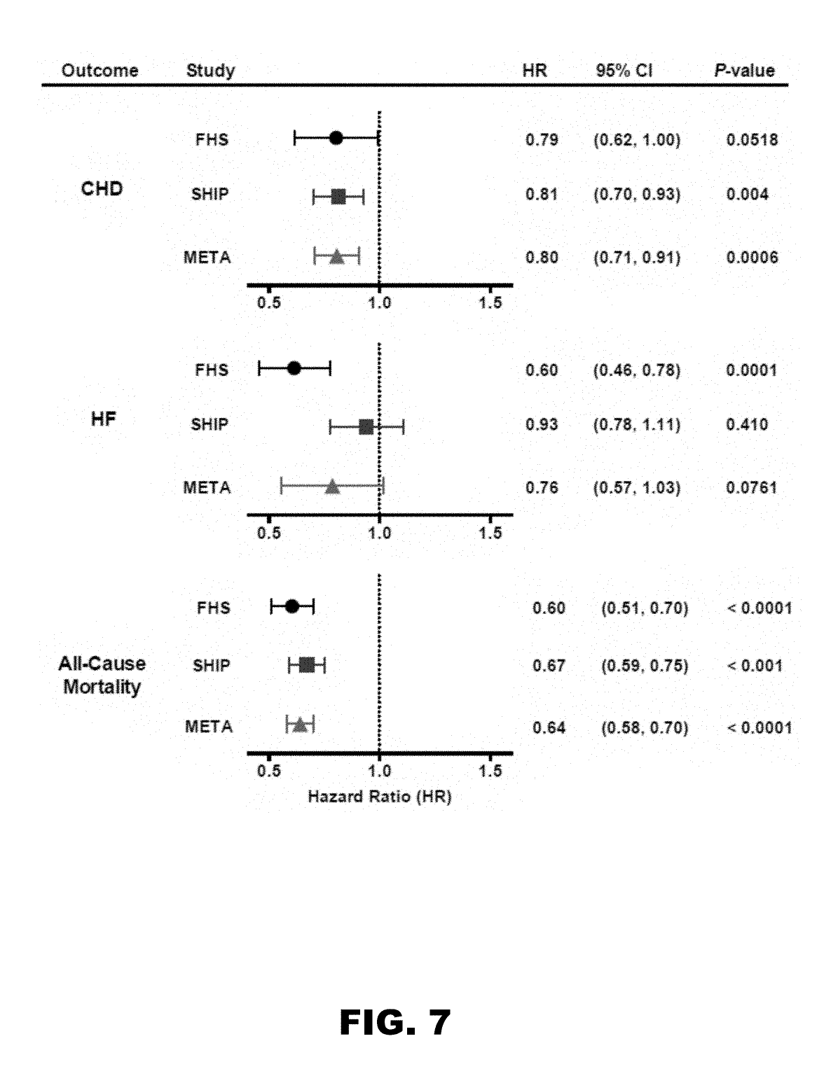

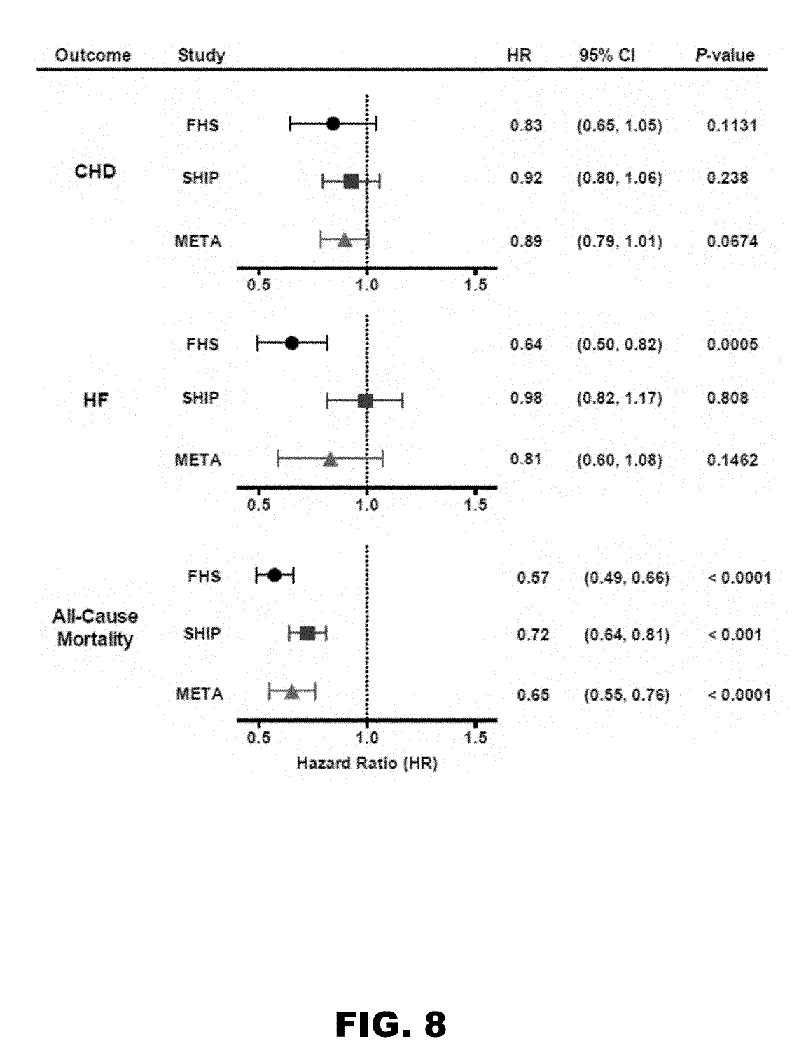

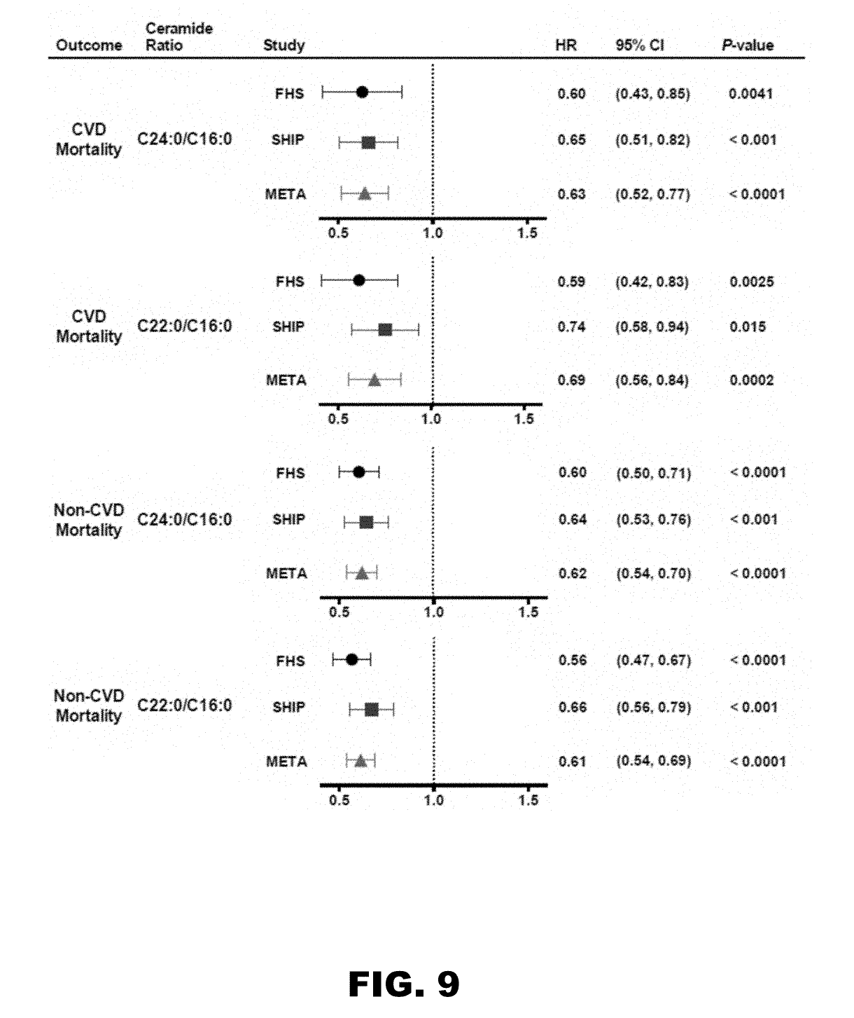

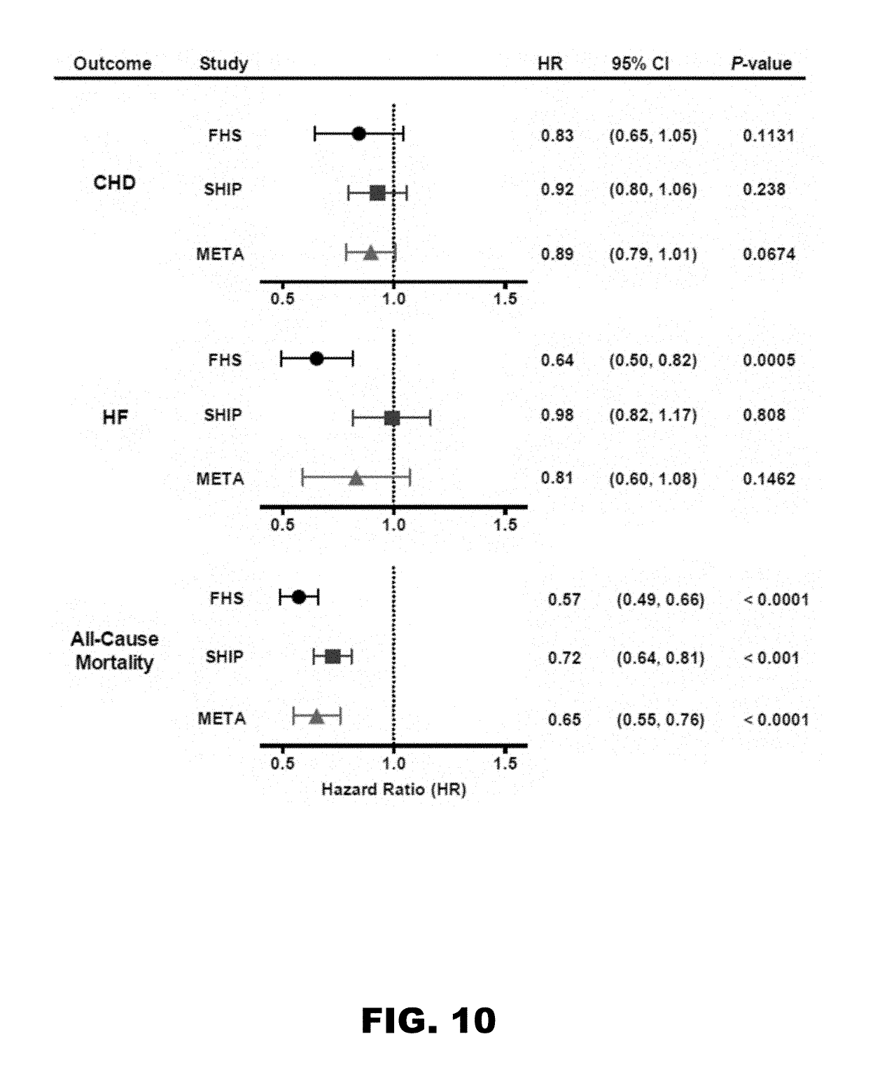

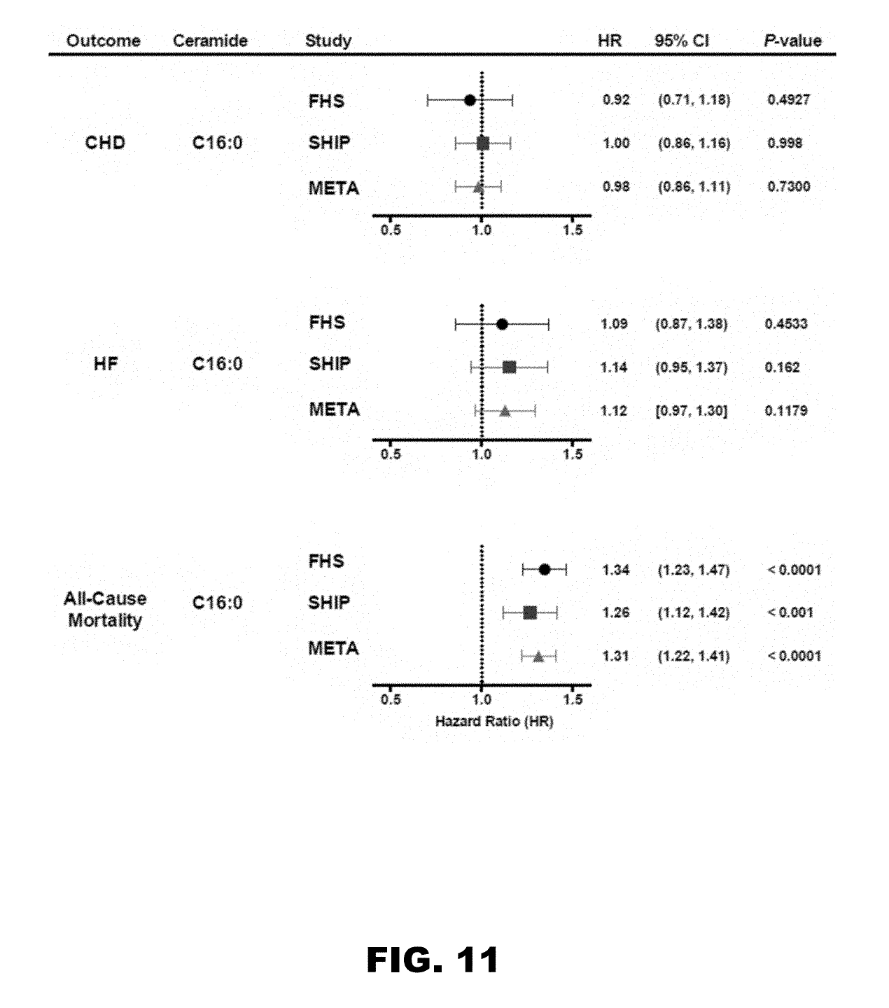

Multivariable-adjusted risk of CHF, HF, and mortality was estimated separately in FHS and SHIP through use of the survival models as described above and then combined through meta-analysis. In the meta-analysis, a significant inverse association between C24:0/C16:0 ceramide ratio and incident CHD was found, with very little variability in effect size due to the between study variation (see e.g., FIG. 7). This inverse association was significant in the SHIP study and of borderline significance in FHS (P=0.0518). The association between C24:0/C16:0 ceramide ratio and incident HF did not reach significance in meta-analyses likely due to high heterogeneity, as indicated by I²>75%, but was inversely associated in FHS. Similar, but weaker trends were observed for association between C22:0/C16:0 ceramide ratio and incident CHD and HF (see e.g., FIG. 8). Most striking in the meta-analysis was an inverse association between the C24:0/C16:0 ceramide ratio and all-cause mortality (see e.g., FIG. 7). This relationship was also observed in each study analyzed individually and reflected inverse associations with both CVD mortality and non-CVD mortality (see e.g., FIG. 9). Associations between the C22:0/C16:0 ratio and mortality (all-cause, CVD, and non-CVD) were similar in the meta-analysis, although the proportion of total variability in effect size due to the between study variation was high for this ratio, as indicated by the I²-statistics. Multivariable-adjusted analyses for individual ceramide species suggests that the findings for the C24:0/C16:0 ceramide ratio were driven by a significant inverse associations between C24:0 ceramide and incident CHD and all-cause mortality (see e.g., FIG. 10). A direct association between C16:0 ceramide and all-cause mortality may have also contributed (see e.g., FIG. 11).

To assess the predictive value of the C24:0/C16:0 and C22:0/C16:0 ceramide ratios, the ceramide ratios from FHS and SHIP were added to base models including standard coronary risk factors of age, sex, BMI, Total/HDL cholesterol, lipid-lowering medications, SBP, antihypertensive therapy, diabetes, and smoking. Addition of the C24:0/C16:0 ceramide ratio did not affect the c-statistic for CHD or HF (see e.g., TABLE 6).

TABLE 6
Incremental Effect of Incorporating Ceramide Ratios on Model Discrimination

| | FHS Samples | | | | |
|---|---|---|---|---|---|
| | Incident | Incident | All-Cause | CVD | Non-CVD |
| | CHD | HF | Mortality* | Mortality* | Mortality* |
| Predictors in Model | c-statistic | c-statistic | c-statistic | c-statistic | c-statistic |
| Standard Risk Factors (SRF) ^{†} | 0.703 | 0.839 | 0.756 | 0.756 | 0.743 |
| SRF + C22:0/C16:0 ratio | 0.717 | 0.844 | 0.776^{‡} | 0.776^{‡} | 0.768^{‡} |
| SRF + C24:0/C16:0 ratio | 0.714 | 0.844 | 0.774^{‡} | 0.774^{‡} | 0.765^{‡} |
| | SHIP Samples | | | | |
| | Incident | Incident | All-Cause | CVD | Non-CVD |
| | CHD | HF | Mortality* | Mortality* | Mortality* |
| Predictors in Model | c-statistic | c-statistic | c-statistic | c-statistic | c-statistic |
| Standard Risk Factors (SRF) ^{†} | 0.7156 | 0.6699 | 0.8625 | 0.9181 | 0.8513 |
| SRF + C22:0/C16:0 ratio | 0.7161 | 0.6704 | 0.8684^{‡} | 0.9203 | 0.8575 |
| SRF + C24:0/C16:0 ratio | 0.7214 | 0.6718 | 0.8695^{‡} | 0.9222 | 0.8584 |

*Prevalent CVD added to standard risk factors
^{†} Standard risk factors: age, sex, BMI, SBP, antihypertensive medication, current smoking status, diabetes, total/HDL cholesterol, and lipid-lowering medication
^{‡}Confidence interval for change in c-statistic excludes 0
FHS = Framingham Heart Study
SHIP = Study of Health in Pomerania

However, addition of the C24:0/C16:0 ceramide ratio improved the c-statistic for all-cause mortality from 0.756 to 0.774 in FHS and from 0.8625 to 0.8695 in SHIP. The confidence interval for the change in c-statistic for both studies excluded 0. In FHS only, addition of the C24:0/C16:0 ceramide ratio also improved the c-statistic for CVD mortality and for non-CVD mortality. Overall, similar improvements in the c-statistic were observed for the C22:0/C16:0 ceramide ratio.

Discussion and Conclusions

The ratio of C24:0/C16:0 ceramides in blood have been shown to be a valuable biomarker of coronary heart disease risk and all-cause mortality in the community.

In two large community-based observational studies, it was observed that higher plasma C24:0/C16:0 ceramide ratios are associated with lower rates of incident CHD and all-cause mortality over a mean follow up of approximately 6 years. Meta-analyses estimated that for every standard deviation increase in plasma C24:0/C16:0 ratio, there was a 20% lower hazard of developing clinical CHD and a 36% lower hazard of all-cause mortality in multivariable adjusted models. A similar inverse association for C22:0/C16:0 ceramide ratio and all-cause mortality was found. Consistent with these observations, it was noted that the C24:0/C16:0 ceramide ratio correlated inversely with several known risk factors for CVD and with prevalent CVD in both FHS and SHIP. The present disclosure provides the first demonstration that the ratio of very long chain to long chain ceramide molecular species in plasma is an independent predictor of CHD and mortality risk in the general population.

The present findings were unanticipated, since prior observations that total plasma ceramides are directly associated with CVD risk factors suggested that plasma ceramides reflect changes in lipid metabolism that promote CHD and mortality.^{14, 15, 33} Instead, in both FHS and SHIP, the C24:0/C16:0 ceramide ratio was inversely associated with the coronary risk factors of age and smoking status and also inversely associated with prevalent CVD. The present findings show that C24:0/C16:0 ratios are lower in men with CHD compared to controls. Importantly, these results indicate that this relation to prevalent CHD is generalizable to community-based samples. While the inverse association of the ceramide ratio with incident CHD was significant in meta-analyses and in analysis of SHIP alone (after adjusting for established coronary risk factors), the association was of borderline significance in FHS alone. Inverse association with incident HF was observed in FHS but not in SHIP, and this association was not statistically significant in the meta-analyses. Even though the definitions of CHD and HF in the analyses of SHIP mirrored those used in FHS, FHS employs continuous surveillance for these events, whereas CHD and HF were ascertained at time of follow-up examinations in SHIP. Thus, one potential reason for the differences in associations for these outcomes between the two studies may be differences in their follow-up methods.

On the other hand, ascertainment of all-cause mortality is an endpoint that is less likely to be affected by methodological differences between studies. The meta-analysis revealed a strong inverse association of the C24:0/C16:0 and C22:0/C16:0 ratios with all-cause mortality—even after adjusting for established coronary risk factors—that reflected significant inverse associations in both FHS and SHIP. Inverse association of the ratios with CVD mortality is consistent with inverse associations with coronary risk factors and prevalent CVD. However, reasons for strong inverse associations of the ceramide ratios with non-CVD mortality will require future investigation. In FHS and in SHIP, both ceramide ratios favorably impacted the c-statistic for all-cause mortality, providing incremental information after adjustment for standard CVD risk factors. Previous retrospective case-control studies showed that the C24:0/C16:0 ceramide ratio is inversely related to CVD mortality among CHD patients. The present disclosure demonstrates that quantification of plasma C24:0/C16:0 or C22:0/C16:0 ratios provide prognostic information in the general population that includes both men and women free of prevalent CVD and over a broad age range. Furthermore, the present observations indicate that lower circulating levels of these very long chain ceramides can antedate the clinical CVD events by several years.

Addition of the C24:0/C16:0 ceramide ratio to model fit for mortality has a modest effect (˜2%), as quantified by area under the receiver-operator characteristic curve or change in c-statistic. However, even accepted measures in cardiovascular risk prediction that are widely used in clinical practice confer small changes in the c-statistic when considered individually, reflecting the relative insensitivity of the c-statistic as a metric for risk prediction in prospective cohorts of healthy individuals. Adding the ceramide ratio as a predictor of mortality in the FHS and SHIP populations compares favorably with the effects of adding high sensitivity C-reactive protein (hsCRP) or B-type natriuretic peptide (BNP) to predictive models, both of which are broadly used in clinical settings. As a marker of systemic inflammation, hsCRP was important in motivating the current CIRT and CANTOS trials, studies that are testing treatments that target systemic inflammation to decrease cardiovascular risk. In an analogous manner, remodeling of ceramide molecular species has the potential to inform about biology not captured in traditional risk factors and thus, potential for identification of novel pathways for targeting treatment. Emerging data also suggests that the ceramide ratio may improve risk prediction in individuals considered at intermediate global risk by ATP-III criteria.

The changes in the relative distribution of acyl chains among plasma ceramides reflects remodeling of the plasma lipidome. In general, the findings for the C24:0/C16:0 ratio in plasma paralleled the biology of C24:0 ceramide, which is the most abundant circulating molecular species. The inverse association of the ratio with all-cause mortality may also reflect in part the direct association between circulating C16:0 ceramide concentrations and this outcome. Overall, the stronger associations for the C24:0/C16:0 ratio compared to those for the C22:0/C16:0 ratio suggests a potentially greater biological significance of the more abundant of the two very long chain species in circulation. Although ceramides are secreted from hepatocytes and generated extracellularly by secreted sphingomyelinases, the knowledge regarding the regulation of plasma ceramide levels is quite limited. Future studies will be performed to elucidate the relation of plasma ratios to ceramide content in specific tissues and how this may impact the pathogenesis of vascular disease and related outcomes.

These results expand the understanding of ceramide biology in several ways. Described herein is a robust methodology to simultaneously quantify the most abundant circulating very long chain and long chain ceramides in large community-based samples under longitudinal surveillance for the development of CVD events. The present findings indicate that ceramide ratios can be precisely and accurately quantified in stored plasma samples and can provide predictive information regarding both CHD and mortality years before the actual onset of disease.

It is envisioned to use these methods in studies to investigate basic questions regarding how tissue and plasma ceramide ratios are regulated and how generalizable these finding from two largely white sample sets may be to other races and ethnic groups. Application of the novel assay for ceramide ratios to larger numbers of multi-ethnic individuals, to those with prevalent CVD, and to samples that have been followed for longer periods will further elucidate the potential utility of this biomarker for mortality risk stratification.

### REFERENCES

1. Hannun Y A and Obeid L M. Many ceramides. J Biol Chem. 2011; 286:27855-62.
2. Park J W, Park W J and Futerman A H. Ceramide synthases as potential targets for therapeutic intervention in human diseases. Biochim Biophys Acta. 2014; 1841:671-81.
3. Holland W L, Brozinick J T, Wang L P, Hawkins E D, Sargent K M, Liu Y, Narra K, Hoehn K L, Knotts T A, Siesky A, Nelson D H, Karathanasis S K, Fontenot G K, Birnbaum M J and Summers S A. Inhibition of ceramide synthesis ameliorates glucocorticoid-, saturated-fat-, and obesity-induced insulin resistance. Cell Metab. 2007; 5:167-79.
4. Li Z, Zhang H, Liu J, Liang C P, Li Y, Li Y, Teitelman G, Beyer T, Bui H H, Peake D A, Zhang Y, Sanders P E, Kuo M S, Park T S, Cao G and Jiang X C. Reducing plasma membrane sphingomyelin increases insulin sensitivity. Mol Cell Biol. 2011; 31:4205-18.
5. Bruce C R, Risis S, Babb J R, Yang C, Kowalski G M, Selathurai A, Lee-Young R S, Weir J M, Yoshioka K, Takuwa Y, Meikle P J, Pitson S M and Febbraio M A. Overexpression of sphingosine kinase 1 prevents ceramide accumulation and ameliorates muscle insulin resistance in high-fat diet-fed mice. Diabetes. 2012; 61:3148-55.
6. Bikman B T and Summers S A. Ceramides as modulators of cellular and whole-body metabolism. J Clin Invest. 2011; 121:4222-30.
7. Park T S, Panek R L, Mueller S B, Hanselman J C, Rosebury W S, Robertson A W, Kindt E K, Homan R, Karathanasis S K and Rekhter M D. Inhibition of sphingomyelin synthesis reduces atherogenesis in apolipoprotein E-knockout mice. Circulation. 2004; 110:3465-71.
8. Park T S, Hu Y, Noh H L, Drosatos K, Okajima K, Buchanan J, Tuinei J, Homma S, Jiang X C, Abel E D and Goldberg I J. Ceramide is a cardiotoxin in lipotoxic cardiomyopathy. J Lipid Res. 2008; 49:2101-12.
9. Russo S B, Baicu C F, Van Laer A, Geng T, Kasiganesan H, Zile M R and Cowart L A. Ceramide synthase 5 mediates lipid-induced autophagy and hypertrophy in cardiomyocytes. J Clin Invest. 2012; 122:3919-30.
10. Chakraborty M, Lou C, Huan C, Kuo M S, Park T S, Cao G and Jiang X C. Myeloid cell-specific serine palmitoyltransferase subunit 2 haploinsufficiency reduces murine atherosclerosis. J Clin Invest. 2013; 123:1784-97.
11. Amati F, Dube J J, Alvarez-Carnero E, Edreira M M, Chomentowski P, Coen P M, Switzer G E, Bickel P E, Stefanovic-Racic M, Toledo F G and Goodpaster B H. Skeletal muscle triglycerides, diacylglycerols, and ceramides in insulin resistance: another paradox in endurance-trained athletes? Diabetes. 2011; 60:2588-97.
12. Luukkonen P K, Zhou Y, Sadevirta S, Leivonen M, Arola J, Oresic M, Hyotylainen T and Yki-Jarvinen H. Hepatic ceramides dissociate steatosis and insulin resistance in patients with non-alcoholic fatty liver disease. J Hepatol. 2016; 64:1167-75.
13. Blachnio-Zabielska A U, Koutsari C, Tchkonia T and Jensen M D. Sphingolipid content of human adipose tissue: relationship to adiponectin and insulin resistance. Obesity (Silver Spring). 2012; 20:2341-7.
14. Boon J, Hoy A J, Stark R, Brown R D, Meex R C, Henstridge D C, Schenk S, Meikle P J, Horowitz J F, Kingwell B A, Bruce C R and Watt M J. Ceramides contained in LDL are elevated in type 2 diabetes and promote inflammation and skeletal muscle insulin resistance. Diabetes. 2013; 62:401-10.
15. de Mello V D, Lankinen M, Schwab U, Kolehmainen M, Lehto S, Seppanen-Laakso T, Oresic M, Pulkkinen L, Uusitupa M and Erkkila A T. Link between plasma ceramides, inflammation and insulin resistance: association with serum IL-6 concentration in patients with coronary heart disease. Diabetologia. 2009; 52:2612-5.
16. Liang H, Tantiwong P, Sriwijitkamol A, Shanmugasundaram K, Mohan S, Espinoza S, Defronzo R A, Dube J J and Musi N. Effect of a sustained reduction in plasma free fatty acid concentration on insulin signalling and inflammation in skeletal muscle from human subjects. J Physiol. 2013; 591:2897-909.
17. Bergman B C, Brozinick J T, Strauss A, Bacon S, Kerege A, Bui H H, Sanders P, Siddall P, Wei T, Thomas M K, Kuo M S and Perreault L. Muscle sphingolipids during rest and exercise: a C18:0 signature for insulin resistance in humans. Diabetologia. 2016; 59:785-98.
18. Spijkers L J, van den Akker R F, Janssen B J, Debets J J, De Mey J G, Stroes E S, van den Born B J, Wijesinghe D S, Chalfant C E, MacAleese L, Eijkel G B, Heeren R M, Alewijnse A E and Peters S L. Hypertension is associated with marked alterations in sphingolipid biology: a potential role for ceramide. PLoS One. 2011; 6:e21817.
19. Chokshi A, Drosatos K, Cheema F H, Ji R, Khawaja T, Yu S, Kato T, Khan R, Takayama H, Knoll R, Milting H, Chung C S, Jorde U, Naka Y, Mancini D M, Goldberg I J and Schulze P C. Ventricular assist device implantation corrects myocardial lipotoxicity, reverses insulin resistance, and normalizes cardiac metabolism in patients with advanced heart failure. Circulation. 2012; 125:2844-53.
20. Pan W, Yu J, Shi R, Yan L, Yang T, Li Y, Zhang Z, Yu G, Bai Y, Schuchman E H, He X and Zhang G. Elevation of ceramide and activation of secretory acid sphingomyelinase in patients with acute coronary syndromes. Coron Artery Dis. 2014; 25:230-5.
21. Baranowski M, Blachnio-Zabielska A, Hirnle T, Harasiuk D, Matlak K, Knapp M, Zabielski P and Gorski J. Myocardium of type 2 diabetic and obese patients is characterized by alterations in sphingolipid metabolic enzymes but not by accumulation of ceramide. J Lipid Res. 2010; 51:74-80.
22. Kasumov T, Solomon T P, Hwang C, Huang H, Haus J M, Zhang R and Kirwan J P. Improved insulin sensitivity after exercise training is linked to reduced plasma C14:0 ceramide in obesity and type 2 diabetes. Obesity (Silver Spring). 2015; 23:1414-21.
23. Serlie M J, Allick G, Groener J E, Ackermans M T, Heijligenberg R, Voermans B C, Aerts J M, Meijer A J and Sauerwein H P. Chronic treatment with pioglitazone does not protect obese patients with diabetes mellitus type I I from free fatty acid-induced insulin resistance. J Clin Endocrinol Metab. 2007; 92:166-71.
24. Warshauer J T, Lopez X, Gordillo R, Hicks J, Holland W L, Anuwe E, Blankfard M B, Scherer P E and Lingvay I. Effect of pioglitazone on plasma ceramides in adults with metabolic syndrome. Diabetes Metab Res Rev. 2015; 31:734-44.
25. Raichur S, Wang S T, Chan P W, Li Y, Ching J, Chaurasia B, Dogra S, Ohman M K, Takeda K, Sugii S, Pewzner-Jung Y, Futerman A H and Summers S A. CerS2 haploinsufficiency inhibits beta-oxidation and confers susceptibility to diet-induced steatohepatitis and insulin resistance. Cell Metab. 2014; 20:687-95.
26. Turpin S M, Nicholls H T, Willmes D M, Mourier A, Brodesser S, Wunderlich C M, Mauer J, Xu E, Hammerschmidt P, Bronneke H S, Trifunovic A, LoSasso G, Wunderlich F T, Kornfeld J W, Bluher M, Kronke M and Bruning J C. Obesity-induced CerS6-dependent C16:0 ceramide production promotes weight gain and glucose intolerance. Cell Metab. 2014; 20:678-86.
27. Tarasov K, Ekroos K, Suoniemi M, Kauhanen D, Sylvanne T, Hurme R, Gouni-Berthold I, Berthold H K, Kleber M E, Laaksonen R and Marz W. Molecular lipids identify cardiovascular risk and are efficiently lowered by simvastatin and PCSK9 deficiency. J Clin Endocrinol Metab. 2014; 99:E45-52.
28. Laaksonen R, Ekroos K, Sysi-Aho M, Hilvo M, Vihervaara T, Kauhanen D, Suoniemi M, Hurme R, Marz W, Scharnagl H, Stojakovic T, Vlachopoulou E, Lokki M L, Nieminen M S, Klingenberg R, Matter C M, Hornemann T, Juni P, Rodondi N, Raber L, Windecker S, Gencer B, Pedersen E R, Tell G S, Nygard O, Mach F, Sinisalo J and Luscher T F. Plasma ceramides predict cardiovascular death in patients with stable coronary artery disease and acute coronary syndromes beyond LDL-cholesterol. Eur Heart J. 2016; 37:1967-76.
29. Quehenberger O, Armando A M, Brown A H, Milne S B, Myers D S, Merrill A H, Bandyopadhyay S, Jones K N, Kelly S, Shaner R L, Sullards C M, Wang E, Murphy R C, Barkley R M, Leiker T J, Raetz C R, Guan Z, Laird G M, Six D A, Russell D W, McDonald J G, Subramaniam S, Fahy E and Dennis E A. Lipidomics reveals a remarkable diversity of lipids in human plasma. J Lipid Res. 2010; 51:3299-305.
30. Kannel W B, Wolf P A and Garrison R J. Some risk factors related to the annual incidence of cardiovascular disease and death in pooled repeated biennial measurements: Framingham Heart Study, 30 year follow-up. 1987. Health and Human Services, Bethesda, Md., Publication NIH 87-2703.
31. Dorr M, Wolff B, Robinson D M, John U, Ludemann J, Meng W, Felix S B and Volzke H. The association of thyroid function with cardiac mass and left ventricular hypertrophy. J Clin Endocrinol Metab. 2005; 90:673-7.
32. Jiang H, Hsu F F, Farmer M S, Peterson L R, Schaffer J E, Ory D S and Jiang X. Development and validation of LC-MS/MS method for determination of very long acyl chain (C22:0 and C24:0) ceramides in human plasma. Anal Bioanal Chem. 2013; 405:7357-65.
33. Haus J M, Kashyap S R, Kasumov T, Zhang R, Kelly K R, Defronzo R A and Kirwan J P. Plasma ceramides are elevated in obese subjects with type 2 diabetes and correlate with the severity of insulin resistance. Diabetes. 2009; 58:337-43.
34. Wilson P W, D'Agostino R B, Levy D, Belanger A M, Silbershatz H and Kannel W B. Prediction of coronary heart disease using risk factor categories. Circulation. 1998; 97:1837-47.
35. Cook N R. Use and misuse of the receiver operating characteristic curve in risk prediction. Circulation. 2007; 115:928-35.
36. Ridker P M. C-reactive protein and the prediction of cardiovascular events among those at intermediate risk: moving an inflammatory hypothesis toward consensus. J Am Coll Cardiol. 2007; 49:2129-38.
37. Melander O, Newton-Cheh C, Almgren P, Hedblad B, Berglund G, Engstrom G, Persson M, Smith J G, Magnusson M, Christensson A, Struck J, Morgenthaler N G, Bergmann A, Pencina M J and Wang T J. Novel and conventional biomarkers for prediction of incident cardiovascular events in the community. JAMA. 2009; 302:49-57.
38. Ridker P M. Moving beyond JUPITER: will inhibiting inflammation reduce vascular event rates? Curr Atheroscler Rep. 2013; 15:295.
39. Iqbal J, Walsh M T, Hammad S M, Cuchel M, Tarugi P, Hegele R A, Davidson N O, Rader D J, Klein R L and Hussain M M. Microsomal Triglyceride Transfer Protein Transfers and Determines Plasma Concentrations of Ceramide and Sphingomyelin but Not Glycosylceramide. J Biol Chem. 2015; 290:25863-75.
40. Merrill A H, Jr., Lingrell S, Wang E, Nikolova-Karakashian M, Vales T R and Vance D E. Sphingolipid biosynthesis de novo by rat hepatocytes in culture. Ceramide and sphingomyelin are associated with, but not required for, very low density lipoprotein secretion. J Biol Chem. 1995; 270:13834-41.
41. Schissel S L, Schuchman E H, Williams K J and Tabas I. Zn2+-stimulated sphingomyelinase is secreted by many cell types and is a product of the acid sphingomyelinase gene. J Biol Chem. 1996; 271:18431-6.
42. Marathe S, Schissel S L, Yellin M J, Beatini N, Mintzer R, Williams K J and Tabas I. Human vascular endothelial cells are a rich and regulatable source of secretory sphingomyelinase. Implications for early atherogenesis and ceramide-mediated cell signaling. J Biol Chem. 1998; 273:4081-8.
43. Jiang H, Hsu F F, Farmer M S, Peterson L R, Schaffer J E, Ory D S and Jiang X. Development and validation of LC-MS/MS method for determination of very long acyl chain (C22:0 and C24:0) ceramides in human plasma. Anal Bioanal Chem. 2013; 405:7357-65.
44. US Department of Health and Human Services FaDA, Center for Drug Evaluation and Research and Center for Veterinary Medicine. Guidance for Industry: Bioanalytical Method Validations. 2001; 2015.

### Example 2. Pancreatic Cancer Study

All patients with pancreatic ductal adenocarcinoma enrolled in two recent trials with available baseline plasma or serum were included. One study was a Phase 1 trial of zoledronic acid as neo-adjuvant, perioperative therapy in patients with non-metastatic, resectable pancreatic adenocarcinoma (ZMA). The other study was an open-label, dose-finding, non-randomised, phase 1b study of CCR2 inhibition in combination with FOLFIRONOX in treatment-naïve patients with borderline resectable or locally advanced biopsy-proven pancreatic ductal adenocarcinoma (FOLF).

Patient Characteristics

There were 66 patients total (47 from FOLF, 19 from ZMA). The mean age of the patients was 62.2 with 46.3% women. Over a median follow-up period of 1.43 years (95% CI 0.98-2.60), there were 52 deaths (78.8%).

The patients in the study of the predictive value of ceramides were previously described in a study of Zoledronic Acid or in a study of CCR2 inhibition plus FOLFIRINOX. The patients in this study had baseline blood samples drawn before treatment in both studies. The blood samples were all centrifuged to obtain the plasma, which was then stored at −80 degrees Fahrenheit until they could be analyzed using the same exact LC/MS-MS techniques outlined in the study of the subjects from the Framingham and SHIP cohorts, Example 1.

The characteristics of the patients and inclusion/exclusion criteria for participation in: A Study of Zoledronic Acid as a Neo-adjuvant, perioperative therapy in patients with resectable pancreatic ductal adenocarcinoma” are published in J Cancer Ther 2013; 4(3):797-803. Of note, treatment with Zoledronic Acid (ZA) did not change overall survival, or progression-free-survival “compared with historical controls” (Stage 2B, N=455). A brief description of subject characteristics is as follows (all quotations regarding the patients are from this published paper): “All patients provided informed, written consent and were treated and Barnes-Jewish Hospital/Washington University Medical Center. Biopsy-proven PDAC patients with tumors that appeared amenable to surgical resection based on pre-operative imaging were eligible for this study. Patients underwent blood draw and bone marrow biopsy at baseline and then received 4 mg IV of Zoledronic Acid (Zometa, Novartis)”. “Patients with newly diagnosed, histologically or cytologically confirmed diagnosis of resectable pancreatic adenocarcinoma who were candidates for surgical treatment were eligible for this study. The eligibility criteria were defined as follows: measurable or evaluable disease defined by RECIST criteria; >18 years old; Karnofsky Performance Status (KPS) 70; life expectancy >12 weeks; adequate bone marrow functions defined as an absolute neutrophil count >1,500/mm3, platelet count >100,000/mm3 and hemoglobin >10 g/dL; adequate renal function defined as serum creatinine 1.3 mg/dL or creatinine clearance 90 mg/min/1.73 m2 with a serum creatinine>1.3 mg/d1; adequate hepatic function defined as total bilirubin 1.5× the institutional upper limit normal value (ULN) after relieving biliary obstruction and aspartate aminotransferase (AST) 2× the ULN. The following patients were excluded from the study: pregnant patients, patients with prior or current autoimmune disease, HIV+ patients, patients receiving other investigational drugs, patient treated with a bisphosphonate within the previous 6 month, patient with current active dental problems.”

“All patients had T3 N1 M0 (Stage 2B cancer)”. “All patients who received at least 1 does of ZA were followed up for survival. Patients followed up with a physician 1, 3, and 6 months after surgery and routine labs were obtained at each visit. Although tumor response was not the primary endpoint of this trial, subjects were monitored for recurrence during the event monitoring period, as clinically indicated. Measurable disease was assessed by the Response Evaluation Criteria in Solid Tumor (RECIST) 1.1.” Overall survival rates are shown in the published paper listed above.

Other plasma samples in our ceramides study were from patients with pancreatic cancer, who were enrolled in the Phase 1b study targeting tumour associated macrophages with CCR2 inhibition plus FOLFIRINOX in locally advanced and borderline resectable pancreatic cancer Lancet Oncol. 2016 May; 17(5): 651-662. PMCID: PMC540728 NIHMSID: NIHMS855337 PMID: 27055731 In this trial, a “single-center, open label, phase 1b clinical trial patients age 18 years with treatment naïve borderline resectable or locally advanced, biopsy-proven pancreatic ductal adenocarcinoma, Eastern Cooperative Oncology Group performance status <2, measurable disease by Response Evaluation Criteria in Solid Tumors Version 1.1, and normal end organ function were eligible for enrollment. FOLFIRINOX (oxaliplatin, 85 mg/m2; irinotecan, 180 mg/m2; leucovorin, 400 mg/m2, and bolus fluorouracil 400 mg/m2 followed by 2,400 mg/m2 46 hour continuous infusion) was administered every 2 weeks for a total of six treatment cycles. To determine the recommended phase 2 dose, PF-04136309 was orally administered at a starting dose of 500 mg twice daily in a standard 3+3 dose de-escalation design with an expansion phase planned at the recommended phase 2 dose. Both FOLFIRINOX and PF-04136309 were simultaneously initiated with a total treatment duration of 3 months. The primary endpoints were to determine the recommended phase 2 dose and toxicity of PF-04136309 in combination with FOLFIRINOX. All patients in the dose de-escalation and expansion phase received the recommended phase 2 dose of PF-04136309 were combined for assessment of treatment toxicity by an intention to treat analysis.” “No therapy related deaths occurring during the study interval. Early termination as the result of treatment related toxicity occurred in 2 of the 39 patients (5%) in the FOLFIRINOX plus PF-04136309 arm.”

Further patient description in this trial: “No upper age limit was established for enrollment in the study.” “Patients were required to have an Eastern Collaborative Oncology Group (ECOG) performance score of 1 or less and an estimated life expectancy >6 months at time of enrollment. Inclusion criteria required evidence of normal bone marrow function (absolute neutrophil count≥1,500/mcl, platelets≥100,000/mcl, hemoglobin≥9.0 g/dl) and end-organ function (creatinine clearance >60 ml/min, a serum bilirubin less than 1.5× upper limit of normal, and a normal International Normalized Ratio (INR) for patients not on anticoagulant therapy). Baseline laboratory tests were obtained for eligibility screening prior to enrollment”. “Exclusion criteria included any prior or current treatment, evidence of metastasis, duodenal/ampullary adenocarcinoma, neuroendocrine tumor, or a prognosis of survival <6 months. Additional exclusion criteria included pregnancy and a history of malignancy in prior 3 years, excluding basal or squamous cell carcinoma of the skin treated with local excision only or carcinoma in situ of the cervix. Patients taking chronic oral steroids were also excluded from the study, however steroid use for the prophylactic treatment of chemotherapy related nausea and inhaled steroids were permitted. Placement of biliary stents prior to enrollment was allowed if liver function returned to permissible levels for inclusion. Informed consent was obtained for all enrolled patients under an institutional review board (IRB) approved protocol at Washington University School of Medicine (St. Louis, Mo.).” Description of all of the patient characteristics who were in this Folfirinox study are published in the article listed above. This study was not designed to evaluate mortality.

Of note, in the above-described two studies, we did not have serum CA 19-9, a cancer antigen used to diagnose and monitor pancreatic cancer https://emedicine.medscape.com/article/2087513-overview, in all patients, but it was not predictive of mortality (HR=1), unlike the Ceramide 16:0 and Ceramide 24:0/16:0.

Results

In time-to-event analysis, the baseline Ceramide 16 level was associated with increased hazard of death (HR 80.2 per 5 unit increase, 95% CI 2.3-2768.2, p=0.0152) while the ratio of Ceramide 24 to Ceramide 16 was associated with improved survival (HR 0.55 per 5 unit increase, 95% CI 0.32-0.94, p=0.0281).

The lower the Ceramide 24:0/16:0 in patients with pancreatic cancer there is a higher risk of death. The Hazard ratio for every increase of 1 in the Cer 24:16 ratio was 0.896 (95% CI 0.806-0.996, p=0.0417). Including age and gender in the multivariable model the ceramide ratio remains significant as a predictor. Hazard ratio for a unit change of 5 is close to 0.5. Higher age and males are also 2× more likely to die.

TABLE 7
Demographics

| | Study Patients | |
|---|---|---|
| | N = 66 | |
| Trial: | | |
| ZMA, n (%) | 19 | (28.8) |
| FOLF, n (%) | 47 | (71.2) |
| Female, n (%) | 31 | (47.0) |
| Race | | |
| African-American, n (%) | 9 | (13.6) |
| Caucasian, n (%) | 55 | (83.3) |
| Other, n (%) | 2 | (3.0) |
| Age, mean (SD) | 62.2 | (8.6) |
| Follow-up (years), median (Q1, Q3) | 1.43 | (0.98, 2.60) |

TABLE 8
Univariate Hazard of All Cause Mortality

| | |
|---|---|
| HR, 95% CI | p |

| | | | |
|---|---|---|---|
| Ceramide 16 | | | |
| 1 unit increase | 2.4 | (1.2-4.9) | 0.015 |
| 5 unit increase | 80.2 | (2.3-2768.2) | |
| Ceramide 24/16 | | | |
| 1 unit increase | 0.89 | (0.80-0.99) | 0.028 |
| 5 unit increase | 0.55 | (0.32-0.94) | |
| Female | 0.54 | (0.31-0.95) | 0.032 |
| Age, per 10 year increase | 1.45 | (1.1-2.0) | 0.022 |
| Trial (ZMA vs FOLF) | 1.02 | (0.56-1.84) | 0.95 |

TABLE 9
Stepwise Multivariate Proportional-Hazard
of All Cause Mortality with Ceramide 24/16

| | HR, 95% CI | p |
|---|---|---|
| Ceramide 24/16 | | |
| 1 unit increase | 0.88 (0.79-0.98) | 0.022 |
| 5 unit increase | 0.54 (0.32-0.91) | |
| Female | 0.53 (0.30-0.93) | 0.024 |
| Age, per 10 year increase | 1.48 (1.06-2.07) | 0.020 |

*Model built with Ceramide 24/16. Ceramide 16 not included. Trial (ZMA vs FOLF) not included in final model due to lack of significance.

TABLE 10
Stepwise Multivariate Proportional-Hazard
of All Cause Mortality with Ceramide 16

| | |
|---|---|
| HR, 95% CI | p |

| | | | |
|---|---|---|---|
| Ceramide 16 | | | |
| 1 unit increase | 2.7 | (1.3-5.5) | <0.01 |
| 5 unit increase | 139.5 | (3.9-4980.7) | |
| Female | 0.50 | (0.28-0.88) | 0.019 |
| Age, per 10 year increase | 1.51 | (1.07-2.12) | 0.018 |

*Model built with Ceramide 16. Ceramide 24/16 ratio not included. Trial (ZMA vs FOLF) not included in final model due to lack of significance.

What is claimed is:

1. A method of determining a risk of a subject to develop a cardiovascular disease, disorder, or condition or a risk of death of a subject comprising:
(i) measuring an amount of C16:0 and C22:0 or C16:0 and C24:0 in a biological sample obtained from a subject;
(ii) determining if a ratio of C22:0 to C16:0 or a ratio of C24:0 to C16:0 is increased or decreased relative to a reference value; and
(iii) classifying the subject as having an increased risk of death or developing a cardiovascular disease, disorder, or condition if the ratio of C22:0 to C16:0 or C24:0 to C16:0 is decreased relative to the reference value.

2. The method of claim 1, wherein the subject has no other risk factors for CVD.

3. The method of claim 1, wherein the subject has one or more risk factors for CVD.

4. The method of claim 3, wherein the one or more risk factors is selected from the group consisting of male gender, body mass index (BMI), high blood pressure, prior CVD, high cholesterol, and age.

5. The method of claim 1, wherein a decreased C24:0 to C16:0 ratio comprises
(i) a C24:0 to C16:0 ratio less than 13.8; or
(ii) a C24:0 to C16:0 ratio less than a reference value,
wherein a decreased C24:0 to C16:0 ratio indicates increased risk of cardiovascular disease, coronary heart disease, heart failure, cardiovascular mortality, non-cardiovascular mortality, increased risk of death from pancreatic cancer or all-cause mortality; or indicates increased coronary risk factors, optionally, age and smoking status; and the reference value is a healthy human subject C24:0 to C16:0 ratio value.

6. The method of claim 1, wherein a decreased C22:0 to C16:0 ratio comprises
(i) a C22:0 to C16:0 ratio less than 3.7; or
(ii) a decreased C22:0 to C16:0 ratio compared to a reference value,
wherein a decreased C22:0 to C16:0 ratio indicates the subject has an increased risk of cardiovascular disease, coronary heart disease, heart failure, cardiovascular mortality, non-cardiovascular mortality, all-cause mortality, increased systolic blood pressure, or increased total/HDL cholesterol.

7. The method of claim 1, wherein the ceramide levels are measured using tandem mass spectroscopy comprising control samples;
a total number of control samples comprise at least 5% of unknown subject samples;
the biological sample comprises a blood or plasma sample;
the biological sample is collected from a fasting subject;
the risk of cardiovascular death or non-cardiovascular death is determined; or
a therapeutic intervention is performed if a ceramide ratio level indicates an increased risk of a cardiovascular disease, disorder, or condition or death.

8. A method to prevent cardiovascular disease (CVD), disorders, or conditions in a subject, the method comprising:
(a) measuring an amount of C16:0 and C22:0 or C16:0 and C24:0 in a biological sample obtained from a subject;
(b) determining if a ratio of C22:0 to C16:0 or C24:0 to C16:0 is increased or decreased relative to a reference value; and
(c) administering a treatment for the cardiovascular disease, disorder, or condition if the ratio of C22:0 to C16:0 or C24:0 to C16:0 is decreased relative to the reference value.

9. The method of claim 8, wherein the subject has no other risk factors for CVD.

10. The method of claim 8, wherein the subject has one or more risk factors for CVD.

11. The method of claim 10, wherein the one or more risk factors is selected from the group consisting of male gender, body mass index (BMI), high blood pressure, prior CVD, high cholesterol, and age.

12. The method of claim 8, wherein a decreased C24:0 to C16:0 ratio comprises
(i) a C24:0 to C16:0 ratio less than 13.8; or
(ii) a C24:0 to C16:0 ratio less than a reference value,
wherein a decreased C24:0 to C16:0 ratio indicates increased risk of cardiovascular disease, coronary heart disease, heart failure, cardiovascular mortality, non-cardiovascular mortality, increased risk of death from pancreatic cancer or all-cause mortality; or indicates increased coronary risk factors, optionally, age and smoking status; and the reference value is a healthy human subject C24:0 to C16:0 ratio value.

13. The method of claim 8, wherein a decreased C22:0 to C16:0 ratio comprises
(i) a C22:0 to C16:0 ratio less than 3.7; or
(ii) a decreased C22:0 to C16:0 ratio compared to a reference value,
wherein a decreased C22:0 to C16:0 ratio indicates the subject has an increased risk of cardiovascular disease, coronary heart disease, heart failure, cardiovascular mortality, non-cardiovascular mortality, all-cause mortality, increased systolic blood pressure, or increased total/HDL cholesterol.

14. The method of claim 8, wherein the ceramide levels are measured using tandem mass spectroscopy comprising control samples;
a total number of control samples comprise at least 5% of unknown subject samples;
the biological sample comprises a blood or plasma sample;
the biological sample is collected from a fasting subject;
the risk of cardiovascular death or non-cardiovascular death is determined; or
a therapeutic intervention is performed if a ceramide ratio level indicates an increased risk of a cardiovascular disease, disorder, or condition or death.

15. A method for monitoring cardiovascular disease (CVD) in a subject, the method comprising:
(a) measuring an amount of C16:0 and C22:0 or C16:0 and C24:0 in a biological sample obtained from a subject;
(b) determining if a ratio of C22:0 to C16:0 or C24:0 to C16:0 is increased or decreased relative to a reference value; and
(c) then at a later time, measuring the amount of C16:0 and C22:0 or C16:0 and C24:0 sample obtained from the subject, wherein a change in the ratio of C22:0 to C16:0 or C24:0 to C16:0, indicates a change in risk of the subject over time.

16. The method of claim 15, wherein an increase in the ratio of C16:0 and C22:0 or C16:0 and C24:0, indicates an abatement of disease progression and a decrease in the ratio of C16:0 and C22:0 or C16:0 and C24:0, indicates disease progression.

17. The method of claim 15, wherein an increase in the ratio of C16:0 and C22:0 or C16:0 and C24:0, indicates a decreased risk of disease progression and a decrease in the ratio of C16:0 and C22:0 or C16:0 and C24:0, indicates an increased risk of disease progression.

18. The method of claim 16, wherein when disease progression is indicated, the subject is treated.

19. The method of claim 17, wherein an increased risk of disease progression is indicated, the subject is treated.

20. The method of claim 15, wherein the method is used to determine the response to treatment, wherein if the ratio of C22:0 to C16:0 or C24:0 to C16:0, increases, then the subject is responding to treatment and if the ratio of C22:0 to C16:0 or C24:0 to C16:0, decreases or remains the same, then the subject is not responding to treatment.
